(19) United States
(12) Patent Application Publication
(10) Pub. No.: US 2010/0034793 A1
(43) Pub. Date: Feb. 11, 2010

(54) METHOD OF USING STROMA CELLS FROM CORD BLOOD TO EXPAND AND ENGRAFT NUCLEATED CELLS FROM CORD BLOOD

(75) Inventors: Ian McNiece, Luthervilel, MD (US); Jin-Fu Wang, Zhenjiang (CN)

Correspondence Address: EDWARDS ANGELL PALMER & DODGE LLP, P.O. BOX 55874, BOSTON, MA, 02205, US

(73) Assignee: The John Hopkins University, Baltimore, MD (US)

(21) Appl. No.: 11/918,994

(22) PCT Filed: Apr. 19, 2006
(86) PCT No.: PCT/US2006/014915

§ 371 Date: Oct. 26, 2009

Related U.S. Application Data
(60) Provisional application No. 60/673,014, filed on Apr. 19, 2005.
(60) Provisional application No. 60/695,604, filed on Jun. 30, 2005.

(51) Int. Cl. A61K 35/14; C12N 5/077

(52) U.S. Cl. 424/93.71; 435/373; 424/93.7

(57) ABSTRACT

The invention features a method for expanding and engrafting nucleated cells, e.g., progenitor cells, such as hematopoietic cells, obtained from cord blood by co-culturing the nucleated cells with adherent stroma cells, e.g., mesenchymal stem/progenitor cells, also obtained from cord blood.

### RELATED APPLICATIONS

This application claims priority to U.S. Provisional Application Nos.: 60/673,014 filed Apr. 19, 2005, entitled “Ex Vivo Expansion of Cord Blood Mononuclear Cells,” and 60/695,506 filed Jun. 30,2005, entitled “Method Of Using Stroma Cells From Cord Blood To Expand And Engraft Nucleated Cells From Cord Blood,” and both are hereby incorporated by reference in their entirety.

### FIELD

This invention relates to the use of adherent stroma cells from umbilical cord blood to promote the expansion and engraftment nucleated cells also obtained from umbilical cord blood.

### BACKGROUND

A number of U.S. Patents, e.g., U.S. Pat. Nos. 5,486,359; 5,591,625; 5,736,396; 5,811,094; 5,827,740; 5,837,539; 5,908,782; 5,908,784; 5,942,225; 5,965,436; 6,010,696; 6,022,540; 6,087,113; 5,858,390; 5,804,446; 5,846,796; 5,654,186; 6,054,121; 5,827,735; and 5,906,934 disclose mesenchymal stem cells (MSC), which can be differentiated into several progenitor cells, for example muscle progenitor cells, connective tissue cell progenitors, or hepatic oval cells. Muscle progenitor cells differentiate further into cardiac, skeletal as well as smooth muscle cells, whereas the connective tissue cell progenitor may differentiate into bone.

Adult bone marrow-derived MSCs engraft in numerous organs and differentiate along tissue-specific lineages when transplanted into fetal sheep. They enhance engraftment of donor hematopoietic cells after co-transplantation in animal models, and they migrate into areas of muscle degeneration to undergo myogenic differentiation in immunodeficient mice. In humans, bone marrow-derived MSCs have been used to regenerate the marrow microenvironment after myeloablative therapy.

U.S. application Ser. No. 09/985,335 (hereby incorporated by reference), describes somatic stem cells known as unrestricted somatic stem cells (USSCs), which can be derived from human umbilical cord blood, placental blood and/or the blood from a newborn child. USSCs are distinct from but capable of differentiating into mesenchymal stem or progenitor cells, hematopoietic lineage stem or progenitor cells, neural stem or progenitor cells, or endothelial stem or liver progenitor cells. USSCs represent the progenitor of the hematopoietic lineage, the mesenchymal stem cells, as well as neural stem cells. This unique multifunctional capacity and the technology to expand these cells, either as cells that remain stem cells, or as committed cells under distinct differentiation protocols, allows precise characterization, standardization, and utilization of the cells for the production and implementation of stem cell therapy in regenerative medicine.

### SUMMARY

We have determined that adherent stroma cells present in umbilical cord blood (UCB) can be used to support the expansion of nucleated cells also present in UCB. We have also determined that adherent stroma cells obtained from UCB can also be used to increase the engraftment of nucleated cells from UCB in a patient in need thereof.

Accordingly, a first aspect of the invention features a method for culturing nucleated cells from umbilical cord blood (UCB), which involves preparing a co-culture that includes nucleated cells and adherent stroma cells, both of which are obtained from UCB, in which the nucleated cells and adherent stroma cells are added separately to the co-culture.

In an embodiment of the first aspect, the method can also include, concurrently with, intermittently during, or following the culturing of the nucleated cells and the adherent stroma cells, contacting the growth medium with a selection element that includes a plurality of selective binding molecules with specific affinity for the nucleated cells or the adherent stroma cells, so as to select said nucleated cells or adherent stroma cells.

A second aspect of the invention features a method for increasing engraftment of donor nucleated cells in a subject by administering a composition having nucleated cells from umbilical cord blood (UCB) and adherent stroma cells from UCB to the subject.

In an embodiment of the first and second aspects of the invention, the nucleated cells include immature, such as progenitor cells (e.g., hematopoietic stem cells), or mature cells, such as granulocytes, neutrophils, megakaryocytes, macrophages, T cells, natural killer cells, and red blood cells. The nucleated cells also are selected from CD34⁻ cells, CD34⁺ cells, and Lin⁻ cells. In a preferred embodiment, the nucleated cells are CD34⁺, Lin cells. In another preferred embodiment, the nucleated cells are hematopoietic stem cells that make up about 40% of the co-culture.

In another embodiment of the first and second aspects of the invention, the adherent stroma cells include mesenchymal stem cells, e.g., unrestricted somatic stem cells (USSCs). In another embodiment, the USSCs are characterized as being CD13⁺, CD29⁺, CD90⁺, CD105⁺, CD166⁺, SH2⁺, SH3⁺, SH4⁺, CD45⁻, CD34⁻, and CD14⁻. In addition, the USSCs can be further characterized as expressing fibulin-2 and lacking expression of hyaluronan synthase and fibromodulin.

In yet another embodiment of the first and second aspects of the invention, co-culturing the cells yields a greater number of nucleated cells than would be present in a culture of nucleated cells grown in the absence of said adherent stroma cells.

In another embodiment of the first and second aspects, the nucleated cells or the adherent stroma cells are isolated from UCB or derived from cells isolated from UCB.

In another embodiment, the nucleated cells or adherent stroma cells are substantially separated from other cell types prior to culturing.

In yet another embodiment, the nucleated cells or the adherent stroma cells are human cells.

In another embodiment of the first and second aspects of the invention, co-culturing the nucleated cells and adherent stroma cells increases the expansion of the nucleated cells by 2 fold, preferably 10 fold, or 100 fold, more preferably 100 fold or 1,000 fold, and most preferably 1,000,000 fold when the nucleated cells are cultured with the adherent stroma cells for at least one day, preferably one week, more preferably two weeks, and most preferably one month as compared to the increase in expansion of the nucleated cells cultured without the adherent stroma cells for the same time period.

In another embodiment, the nucleated cells (e.g., progenitor cells) are transferred to a separate culture medium following the co-culturing step and cultured under conditions that cause differentiation of the nucleated cells into a pre-determined cell type.

In other embodiments, the nucleated cells or the adherent cells are expanded prior to co-culturing. In yet another embodiment, the co-culturing occurs in the presence of at least one cytokine (e.g., a cytokine selected from granulocyte colony stimulating factor (G-CSF), granulocyte-macrophage colony stimulating factor (GM-CSF), stem cell factor (SCF), interleukin 2 (IL-2), interleukin 3 (IL-3), interleukin 6 (IL-6), thrombopoietin (TPO), FLT-3 ligand, transforming growth factor (TGF)-β, megakaryocyte growth and development factor, tumor necrosis factor α (TNF-α), HOX-B4, and Wnt). In a preferred embodiment, the cytokine is G-CSF.

In another preferred embodiment, the nucleated cells and adherent stroma cells are administered to a patient following the culturing step to treat or prevent a disease or disorder (e.g., a vascular, muscle, hepatic, pancreatic, or neural disease or disorder) or to reconstitute the immune system of the patient. In a preferred embodiment, the nucleated cells are separated from the adherent stroma cells and only the nucleated cells are administered to the patient to treat or prevent the disease or disorder or to reconstitute the immune system of the patient.

In another embodiment, the nucleated cells or the adherent stroma cells are suitable for administration to a human patient.

A third aspect of the invention features a composition that includes nucleated cells and adherent stroma cells, in which nucleated cells and adherent stroma cells are obtained from umbilical cord blood (UCB), and in which the composition is suitable for infusion or engraftment into a subject.

In an embodiment, the adherent stroma cells include mesenchymal stem cells, e.g., USSCs. In another embodiment, the USSCs are characterized as being CD13⁺, CD29⁺, CD90⁺, CD105⁺, CD166⁺, SH2⁺, SH3⁺, SH4⁺, CD45⁻, CD34⁻, and CD14⁻. In addition, the USSCs can be further characterized as expressing fibulin-2 and lacking expression of hyaluronan synthase and fibromodulin.

In another embodiment, the nucleated cells are progenitor cells (e.g., hematopoietic stem cells). In a preferred embodiment, the progenitor cells are selected from CD34⁻ cells, CD34⁺ cells, or Lin⁻ cells. In another preferred embodiment, the progenitor cells are CD34⁺, Lin⁻ cells.

In another embodiment, the nucleated cells or adherent stroma cells are allogeneic or autologous to the subject.

In another embodiment, the nucleated cells or adherent stroma cells are genetically modified.

By “administering” is meant providing to a human patient a pharmaceutical preparation containing the progenitor cells alone, or in combination with USSCs, or their progeny or derivatives in a suitable formulation. The preferred method of administration can vary depending on various factors, e.g., the components of the pharmaceutical preparation, site of the potential or actual disease, and severity of disease.

By “effective amount” is an amount sufficient to effect beneficial or desired clinical or biochemical results. An effective amount can be administered one or more times. For purposes of this invention, an effective amount is the amount of USSCs or combination of donor progenitor cells and USSCs that may be administered to effect beneficial engraftment of the progenitor cells.

By “engraftment” is meant the implantation of cells in the body, and/or replacement of lost or damaged cells with injected cells. The engrafted cells persist in a particular location over time following transplantation of the cells into a mammal (e.g., a human).

By the term “expanded population” is meant a population of cells, e.g., the progenitor cells of the invention, wherein at least 50% of the cells have divided at least once.

A molecule is a “marker” of a desired cell type if it is found on a sufficiently high percentage of cells of the desired cell type, and found on a sufficiently low percentage of cells of an undesired cell type, such that one can achieve a desired level of purification of the desired cell type from a population of cells comprising both desired and undesired cell types by selecting for cells in the population of cells that have the marker. A marker can be displayed on, for example, 30%, 35%, 40%, 45%, 50%, 55%, 60%, 65%, 70%, 75%, 80%, 85%, 90%, 95%, 99% or more of the desired cell type, and can be displayed on fewer than 50%, 45%, 40%, 35%, 30%, 25%, 20%, 15%, 10%,5%, 1% or fewer of an undesired cell type.

A desired cell type is negative for a cell surface-expressed marker or lacks expression of the marker if fewer than 50 marker molecules per cell are present on the cell surface of the desired cell type. Techniques for detecting cell surface-expressed marker molecules are well known in the art and include, e.g., flow cytometry. One skilled in the art can also use enzymatic amplification staining techniques in conjunction with flow cytometry to distinguish between cells expressing a low number of a marker molecule and cells that do not express the marker molecule (see, e.g., Kaplan, Front. Biosci. 7:c33-c43, 2002; Kaplan et al., Amer. J. Clin. Pathol. 116:429-436, 2001; and Zola et al., J. Immunol. Methods 135:247-255, 1990).

By “mesenchymal stem/progenitor cell” is meant an adherent stroma cell from umbilical cord blood that includes mesenchymal stem cells, such as those identified by Wernet (U.S. Patent Application Publication No. 2005/0142118), Caplan et al. (U.S. Pat. No. 5,486,359), Erices et al. (Br. J. Haematology 109: 235-42, 2000), Naughton et al. (U.S. Pat. No. 5,962,325), Hariri et al. (U.S. Patent Application Publication No. 2005/0019908), Weiss et al. (U.S. Patent Application Publication No. 2004/0136967), Baksh et al. (U.S. Patent Application Publication No. 2004/0137612), and Atala et al. (U.S. Patent Application Publication No. 2005/0124003), each of which is incorporated herein by reference.

By a “muscle cell” is meant a skeletal, smooth, or cardiac cell.

By “muscle disease” is meant a disease or disorder that affects or involves the musculature, e.g., cardiac, smooth, or skeletal muscles. Examples of muscle diseases include neuromuscular disease, e.g., muscular dystrophy (e.g., Duchenne muscular dystrophy (DMD), Becker muscular dystrophy (3MD), Limb-girdle muscular dystrophy, and congenital muscular dystrophy), congenital myopathy, and myasthenia gravis, cardiomyopathy, e.g., heart disease, aortic aneurysm (Marfan's disease), cardiac ischemia, congestive heart failure, heart valve disease, and arrhythmia, and metabolic muscle diseases.

By a “neural cell” is meant a neuron (e.g., a sensory neuron, a motor neuron, or an interneuron) or a support cell of the central or peripheral nervous system. Examples of neurons include pyramidal cells, Betz cells, stellate cells, horizontal cells, granule cells, Purkinje cells, spinal motor neurons, and ganglion cells. Examples of support cells include glial cells, oligodendroglial cells, astrocytes, satellite cells, microglial cells, and Schwann cells.

By “neural disease” is meant a disease or disorder that affects or involves the central or peripheral nervous system. Examples of neural diseases include multi-infarct dementia (MID), vascular dementia, cerebrovascular injury, Alzheimer's disease (AD), neurofibromatosis, Huntington's disease, amyotrophic lateral sclerosis, multiple sclerosis, stroke, Parkinson's disease (ED), pathologies of the developing nervous system, pathologies of the aging nervous system, and trauma, e.g., head trauma. Other examples of neural diseases are those that affect tissues of the eye, e.g., the optic stalk, retinal layer, and lens of the eye, and the inner ear. In certain embodiments, the patient may have suffered a neurodegenerative disease, a traumatic injury, a neurotoxic injury, ischemia, a developmental disorder, a disorder affecting vision, an injury or disease of the spinal cord, or a demyelinating disease.

By “progenitor cell” is meant a cell with the capability of multi-lineage differentiation and self-renewal, as well as the capability to regenerate tissue. Although progenitor cells are described mostly with respect to using umbilical cord blood progenitor cells in the present application, the invention is not limited to such and may include progenitor cells of other origin, including but not limited to hematopoietic stem cells, liver stem cells, pancreatic stem cells, neuronal stem cells, bone marrow stem cells, peripheral blood stem cells, umbilical cord blood stem cells or a mixture thereof. Further, the invention is not limited to transplantation of any particular progenitor cell obtained from any particular source, but may include progenitor cells from “multiple stem cell sources” in mixture with one another. Thus, USSCs may be used in cotransplantation of the progenitor cells obtained from single or multiple stem cell sources to increase the amount of engraftment.

By “sample” or “biological sample” is meant any biological sample obtained from an individual, body fluid, cell line, tissue culture, or other source which may contain progenitor cells.

By “stem cell” or “pluripotent stem cell,” which can be used interchangeably, is meant a cell having the ability to give rise to two or more cell types of an organism.

By “subject” is meant a vertebrate, preferably a mammal, more preferably a human.

By “substantially purified” is meant that the desired cells (e.g., progenitor cells and/or USSCs) are enriched by at least 30%, more preferably by at least 50%, even more preferably by at least 75%, and most preferably by at least 90% or even 95%.

By “therapeutically-active protein” is meant a polypeptide that improves or maintains the health of the cell expressing the polypeptide or that of a cell in proximity to the expressing cell. Examples of therapeutically-active proteins include, without limitation, growth factors, cytokines, anti-apoptotic factors, colony stimulating factors, hormones, antiviral proteins, lipocortins, lipotropins, interleukins, interferons, stimulating factors, kinases, cystic fibrosis transmembrane conductance regulators, coagulation factors, immunoglobulins, cell surface proteins, human pancreatic enzymes, and enlcephalins; and in particular: growth hormone (GH; e.g., human growth hormone), interferon (IFN; e.g., IFN-alpha, IFN-beta, or IFN-gamma), albumin, tumor necrosis factor (TNF; e.g., TNF-alpha or TNF-beta), alpha-antitrypsin, hirudin, erythropoietin, thrombopoietin, granulocyte-colony stimulating factor (G-CSF), urokinase, factor VIII, factor IX, arnylase lipase, lactase, human serum albumin, adipokinin, aldosterone, adrenocorticotropin (ACTH), chorionic gonadotropin, corticoliberin, corticotropin, hexokinase, glucokinase, folliberin, follitropin, glucagon gonadoliberin, gonadotropin, hypophysiotropic hormone, insulin, luteinizing hormone-releasing hormone, growth hormone releasing factor, hi stamen releasing factor, luteotropin, melanotropin, parathormone, parotin, prolactin, prolactoliberin, prolactostatin, somatoliberin, somatotropin, thyrotropin, tissue-type plasminogen activator (TPA), Mullerian Inhibiting Substance (MIS), somatostatin, calcitonin, calcitonin gene related peptide (CGRP), β-calcitonin gene related peptide, hypercalcemia of malignancy factor (1-40) (PTH-rP), parathyroid hormone-related protein (107-139) (PTH-rP), parathyroid hormone-related protein (107-111) (PTH-rP), thyroid-stimulating hormone, cholecystokinin, galanin message associated peptide, preprogalanin (65-105), gastrin I, gastrin releasing peptide, glucagon-like peptide (GLP-1), pancreastatin, pancreatic peptide, peptide YY, peptide histidine methionine (PHM), secretin, vasoactive intestinal peptide (VIP), oxytocin, vasotocin, enkephalinamide, metorphinamide (adrenorphin), alpha melanocyte stimulating hormone (alpha-MSH), atrial natriuretic factor (5-28) (ANF), amylin, arnyloid P component (SAP-1), corticotropin releasing hormone (CRH), neuropeptide Y, substance K (neurokinin A), substance P, thyrotropin releasing hormone (TRH), histamine releasing factor, follicle-stimulating hormone, leptin, angiotensin I, angiotensin II, β-endorphin, β-melanocyte stimulating hormone (β-MSH), endothelin I, galanin, gastric inhibitory peptide (GIP), neurophysins, epidermal growth factor, platelet-derived growth factor, fibroblast growth factor, hepatocyte growth factor, insulin-like growth factor 1, GLUT-2, glucagon-like peptide (GLP)-1, insulin promoting factor 1 (IPF1), prohormone convertase 2 (PC2), prohormone convertase 3 (PC3), peptidylglycine alpha-amidating monooxygenase (PAM), glucagon-like peptide I receptor, glucose-dependent insulinotropic polypeptide receptor, baculoviral inhibitor of apoptosis repeat (BIR), sulphonylurea receptor (SUR), growth hormone releasing factor receptor (GHRFR), growth hormone releasing hormone (GHRH), growth hormone releasing hormone receptor (GHRHR), and vasopressin.

By “transgene” is meant any piece of a nucleic acid molecule (for example, DNA) that is inserted by artifice into a cell transiently or permanently, and becomes part of the organism if integrated into the genome or maintained extrachromosomally. Such a transgene may include a gene that is partly or entirely heterologous (foreign) to the transgenic organism, or may represent a gene homologous to an endogenous gene of the organism.

By “transgenic cell” is meant a cell containing a transgene. For example, a cell transformed with an expression vector operably linked to a heterologous nucleic acid molecule can be used to produce a population of cells having altered phenotypic characteristics. A cell derived from a transgenic organism is also a transgenic cell so long as the cells contain the transgene.

By “transplant” or “transplanting” is meant administering one or more cells (or parts thereof), cell products, tissue, or cell culture products derived from cells that are grafted into a human host. Specifically, a transplant is produced by manipulating the cells described herein. These cells can be further manipulated to include heterologous genetic material such as a transgene.

By “treatment” is meant an approach for obtaining beneficial or desired clinical results. For the purposes of this invention, beneficial or desired clinical results include, but are not limited to, alleviation of symptoms, diminishment of extent of disease, stabilization (i.e., not worsening) of a state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment. Typically, the “treatment” entails administering additively effective progenitor cells to the patient to regenerate tissue.

By “umbilical cord blood cells”, “cord blood cells,” or “placental blood cells” we mean the blood that remains in the umbilical cord and placenta following birth. Like bone marrow, cord blood has been found to be a rich source of progenitor and/or stem cells.

By a “vascular cell” is meant an endothelial cell. Endothelial cells line the blood and lymph vessels and are present in and play a key role in the development of organs, such as the brain, heart, liver, pancreas, lungs, spleen, stomach, intestines, and kidneys.

By “vascular disease” is meant a disease or disorder that affects or involves the vasculature. Examples of vascular disease include peripheral vascular disease, peripheral arterial disease, venous disease (e.g., deep vein thrombosis), ischemia, cardiovascular disease, tissue organ engraftment rejection, or sequelae of ischemic reperfusion injury. In still another embodiment, the peripheral vascular disease is atherosclerosis, thromboembolic disease, or Buerger's disease (thromboangiitis obliterans). In a further embodiment, the cardiovascular disease is myocardial infarction, heart disease, or coronary artery disease.

### BRIEF DESCRIPTION OF THE DRAWINGS

FIGS. 1A and 1B are photomicrographs of umbilical cord blood (UCB)-derived adherent cells (×400) after one week (1A) and four weeks (1B) under culture conditions.

FIG. 2 is a graph showing the growth curves of USSCs from UCB. UCB-derived US SCs were plated at a density of 10⁴ cells/cm²; duplicate cultures were harvested every four days for 24 days, and the number of adherent cells was determined. Results are expressed as mean ± SEM (n=5).

FIG. 3 is a graph showing the expansion trends of UCB hematopoietic stem/progenitor cells. —K—: co-culture system with exogenous cytokines; —R—: co-culture system without exogenous cytokines; —G—: control system.

FIG. 4 is a graph showing the expansion of granulocyte-macrophage colony-forming cells (GM-CFC) and high proliferative potential colony-forming cells (HPP-CFC). A: GM-CFC, B: HHPCFC; a: the number of colonies from the starting CD34+ cell fraction; b: the number of colonies from expanded cells in a co-culture system with exogenous cytokines; c: the number of colonies from expanded cells in a co-culture system without exogenous cytokines; and d: the number of colonies from expanded cells in the control system.

FIG. 5 is a photomicrograph showing induced cells stained with toluidine blue (×400). Induced cells secreted a metachromatic matrix that showed positive staining with toluidine blue.

FIG. 6 depicts RT-PCR results with a pair of primers specific for the collagen gene.

FIG. 7 depicts recovery of WBC population in irradiated NOD/SCID mice transplanted with or without expanded HSPCs

FIG. 8 depicts PCR detection of Alu sequence fragment special for human cells in NOD/SCID mice bone marrow and peripheral blood. PCR analysis demonstrated the presence of human hematopoietic cells in the (lane 2) and (lane 3) of NOD/SCID mice transplanted with HSPCs expanded in coculture scheme. DNA extracted from human UCB cells was used as a positive control (lane 4). Negative control (without transplantation) is shown in lane 1 and DNA marker is shown in lane 5.

### DETAILED DESCRIPTION

We have determined that adherent stroma cells present in umbilical cord blood (UCB) can be used to support the expansion of nucleated cells also present in UCB. The adherent stroma cells and the nucleated cells can be prepared separately and added to a co-culture, which results in the expansion of the cells to a greater extent than would be observed if the nucleated cells were cultured alone.

Adherent stroma cells from UCB for use in the invention include mesenchymal stem/progenitor cells (MSPCs), such as those identified by Wernet (U.S. Patent Application Publication No. 2005/0142118), Caplan et al. (U.S. Pat. No. 5,486,359), Erices et al. (Br. J. Haematology 109: 235-42, 2000), Naughton et al. (U.S. Pat. No. 5,962,325), Hariri et al. (U.S. Patent Application Publication No. 2005/0019908), Weiss et al. (U.S. Patent Application Publication No. 2004/0136967), Baksh et al. (U.S. Patent Application Publication No. 2004/0137612), and Atala et al. (U.S. Patent Application Publication No. 2005/0124003), each of which is incorporated herein by reference. Nucleated cells obtained from UCB that can be used in the invention include immature cells (e.g., pluripotent cells, such as hematopoietic stem cells (including, e.g., CD34⁺ or CD34⁻ cells, and Lin⁻ cells)) and mature cells (e.g., granulocytes, neutrophils, megakaryocytes, macrophages, T cells, natural killer cells, and red blood cells).

The adherent stroma cells used in the invention include mesenchymal stem/progenitor cells (MSPCs). MSPCs can be extensively expanded ex vivo and, when cultured under permissive conditions, retain their ability to differentiate into multiple lineages including bone, cartilage, tendon, muscle, nerve, and stroma cells. MSPC are of great therapeutic potential because of their ability to self-renew and differentiate into multiple tissues.

The inventors have determined that adherent stroma cells obtained from UCB, e.g., MSPC, such as USSCs, can support ex vivo expansion of nucleated cells, including progenitor cells, e.g., hematopoietic stem/progenitor cells (HSPC), CD34⁻ cells, CD34⁺ cells, and Lin⁻ cells, and other mature nucleated cells present in UCB, such as granulocytes, neutrophils, megakaryocytes, macrophages, T cells, natural killer cells, and red blood cells. Adherent stroma cells obtained from UCB, such as MSPCs, can also be used to increase the engraftment of nucleated cells present in UCB, e.g., progenitor cells (e.g., hematopoietic stem/progenitor cells (HSPC), CD34⁻ cells, CD34⁺ cells, and Lin⁻ cells) and mature cells (e.g., granulocytes, neutrophils, megakaryocytes, macrophages, T cells, natural killer cells, and red blood cells) when the adherent stroma cells are co-transplanted with the nucleated cells. Finally, MSPCs isolated from human UCB can be readily expanded and induced to differentiate into chrondogenic cells in vitro.

Adherent Stroma Cells Support the Expansion of Nucleated Cells when Co-Cultured

Adherent stroma cells obtained from UCB, e.g., MSPC, such as USSCs, are capable of supporting the ex vivo proliferation and differentiation of nucleated cells obtained from UCB, such as progenitor cells, e.g., hematopoietic stem cells (e.g., CD34⁻ cells, CD34⁺ cells, and Lin⁻ cells), and mature cells, e.g., granulocytes, neutrophils, megakaryocytes, macrophages, T cells, natural killer cells, and red blood cells, when the nucleated cells and adherent stroma cells are co-cultured. Adherent stroma cells support the expansion of nucleated cells when co-cultured in the presence or in the absence of exogenous cytokines (e.g., granulocyte colony stimulating factor (G-CSF), granulocyte-macrophage colony stimulating factor (GM-CSF), stem cell factor (SCF), interleukin 2 (IL-2), interleukin 3 (IL-3), interleukin 6 (IL-6), thrombopoietin (TPO), FLT-3 ligand, transforming growth factor (TGF)-β, megakaryocyte growth and development factor, and tumor necrosis factor α (TNF-α)). In particular, the addition of exogenous G-CSF can enhance the ability of adherent stroma cells to promote expansion of nucleated cells during co-culture. Additional factors that can be included in the co-culture include HOX-B4 and Wnt. The expansion of nucleated cells in co-culture with adherent stroma cells was supported for at least 15 passages of the cells. The co-culture results in at least a 2 fold expansion of the nucleated cells, more preferably a 5 fold, 10 fold, 20 fold, or 50 fold expansion, and most preferably a 100 fold, 1,000 fold, or 1,000,000 fold expansion or more of the nucleated cells over the course of at least one day, more preferably one or two weeks, and most preferably one month, relative to the amount of expansion that occurs over the same period of time in the absence of the adherent stroma cells. Co-culture of the nucleated cells with the adherent stroma cells yields a greater number of expanded cells relative to a culture of the nucleated cells without the adherent stroma cells.

The co-culture of nucleated cells, including, e.g., progenitor cells and mature cells, and adherent stroma cells, such as MSPCs, can be initiated by first preparing a layer of adherent stroma cells in an appropriate container, such as a bioreactor. Once the stroma cells have adhered, the nucleated cells can be added to the container. Alternatively, both the nucleated cells and the adherent stroma cells can be added to the container at the same time and cultured.

Co-culture of the adherent stroma cells with the nucleated cells can also be performed under conditions in which the adherent stroma cells and the nucleated cells are not in direct contact, such as in a transwell plate separated by a semi-permeable membrane. In this embodiment, the cells maintain fluid contact because they are bathed by the same culture medium. Thus, the cells can share growth factors, ECM components, and other secreted factors.

Following co-culture, either the nucleated cells or the adherent stroma cells can be separated to produce a substantially purified population of cells for administration to a patient. Alternatively, the nucleated cells and adherent stroma cells can be administered together. The nucleated cells or the adherent stroma cells can be expanded, either before or after co-culture, to increase their numbers. In cases where the nucleated cells are progenitor cells, such as hematopoietic cells, it is preferred that the progenitor cells not be allowed to differentiate until after expansion in the co-culture. Once the cells have been expanded, they can be separated from the adherent stroma cells and induced to differentiate, or they can be induced to differentiate in the presence of the adherent stroma cells. The cells can also be administered to a patient in an undifferentiated state.

Cotransplanation and Engraftment Using Adherent Stroma Cells from Umbilical Cord Blood

Adherent stroma cells obtained from UCB, e.g., MSPCs, such as USSCs, when co-transplanted with nucleated cells, such as progenitor cells, e.g., hematopoietic cells (e.g., CD34⁻ cells, CD34⁺ cells, and Lin⁻ cells), or mature cells, which are also obtained from UCB, increase the engraftment of the nucleated cells, relative to nucleated cells transplanted without the adherent stroma cells. Adherent stroma cells also exhibit a tolerizing effect on the nucleated cells. Thus, the adherent stroma cells can be used to enhance graft-versus-graft tolerance when using allogeneic donor cells for transplantation. The allogeneic donor cells can be obtained from different sources and combined, and their immunogenic tolerance to each other may be enhanced by cotransplantation with adherent stroma cells.

The immune response of a subject to transplanted donor cells has been a major limiting factor in the number of donor cells that can be administered, although a lower input cell number correlates with higher rates of delayed or failed engraftment. The tolerizing effect of the adherent stroma cells on the donor progenitor cells now allows the transplantation of larger numbers of cells.

Adherent stroma cells, such as MSPCs exert a potent suppressive effect on the allogenic immune response. Thus, graft vs. graft reaction, which normally occurs in the context of donor cell transplantation, can be suppressed by cotransplantation of donor nucleated cells, e.g., progenitor cells and/or mature cells, with adherent stroma cells, e.g., MSPCs. Adherent stroma cells such as MSPCs also promote a higher overall level of engraftment of donor nucleated cells when co-administered. Adherent stroma cells cotransplantation promotes significantly higher engraftment levels of nucleated cells, and these higher levels can be well correlated with more balanced co-engraftment and a display of multipotent lympho-myeloid reconstitution.

### Purification of Adherent Stroma Cells

Adherent stroma cells, e.g., MSPCs, can be isolated and purified as described in U.S. Patent Application Publication No. 2002/0164794, which is incorporated herein by reference. For example, adherent stroma cells can be isolated and purified by the steps of density gradient isolation, culture of adherent cells, and subculture by applying growth factors. After a confluent cell layer has been established, the isolation process to derive adherent stroma cells is controlled by morphology (fibroblastoid morphology) and phenotypical analyses using antibodies directed against CD13 (positive), CD45 (negative), and CD29 (positive) surface antigens.

In an embodiment of the invention, USSCs are the MSPC used for co-culture and engraftment of nucleated and/or progenitor cells. USSCs are negative for markers specific for the hematopoietic lineage such as CD45 and hence are distinct from hematopoietic stem cells, which can also be isolated from placental cord blood. CD14 is another surface antigen that cannot be detected on USSCs. USSCs are further characterized as being positive for a set of antigens which are present on the cell surface such as CD13, CD29, CD44, and CD49e. USSC preparations are further characterized by the presence of MRNA transcripts for certain receptor molecules like epidermal growth factor receptor (EGF-R), platelet derived growth factor receptor alpha (PDGF-RA), and insulin growth factor receptor (IGF-R). These cells also typically express transcription factors such as YB1 (Y-box transcription factor 1), Runx1 (runt related transcription factor 1) and AML1C (acute myeloid leukemia 1 transcription factor) as detected by RT-PCR. USSC preparations are typically negative for transcripts for the chondrogenic transcription factor Cart-1 and neural markers such as neurofilament, synaptophysin, tyrosine hydroxylast (TH) and glial fibriallary acidic protein (GFAP).

TABLE 1
Analysis of the transcription patterns of USSCs by RT PCR

| Name | PCR-Result USSC | PCR-result (other tissue) |
|---|---|---|
| PDGFR_alpha | + | + (adult bone) |
| IGFR | + | + (adult bone) |
| Neurofilament | − | + (adult liver) |
| CD105 | + | + (mononuclear |
| | | cells from CB) |
| GFAP | − | + (fetal brain) |
| Synaptophysin | − | + (fetal brain) |
| Tyrosinhydroxylase | − | + (fetal brain) |
| YB1 | + | + (fetal brain) |
| Runx1 | + | + (adult bone) |
| AML1c | + | + (adult bone) |
| BMPR II | + | + (adult cartilage) |
| Collagen Type I | + | + (adult bone) |
| Cart-1 | − | + (mononuclear |
| | | cells from CB) |
| Chondroadherin | − | + (adult bone) |
| CD49e | + | + (adult bone) |

RT-PCR results achieved with predicted oligonucleotide primers and mRNAs from USSCs and positive control mRNAs from other tissues like bone, cartilage, brain or cord blood mononuclear cells.

The RAN expression of USSC preparations and bone marrow derived MSCs (Caplan, 1991) were directly compared by using quantitative Affymetrix GeneChip™ microarrays. The transcript of the fibulin-2 gene (gene bank number X82494) was detected in USSCs at a high expression levels but not in MSCs. Fibulin-2 production was previously demonstrated in fibroblasts (Pan et al., 1993). Northern blot analysis of MRNA from various human tissues reveals an abundant 4.5 kb transcript in heart, placenta and ovary tissue (Zhang et al., 1994). The protein has been localized at the light microscopical level in human embryos of gestational weeks 4-10, using polyclonal antibodies. Fibulin-2 was detected primarily within the neurophithelium, spinal ganglia and peripheral nerves (Misoge et al., 1996).

In the rat animal model, rat liver myofibroblasts (rMF) are localized with fibulin 2. These cells were located in the portal field, the walls of central veins, and only occasionally in the parenchyma. In early stages of fibrosis rMF were detected within the developing scars. In advanced stages of fibrosis rMF accounted for the majority of the cells located within the scar (Knittel et al., 1999). In an other animal model, mouse Fibulin-2 protein is express during epithelial-mesenchymal transformation in the endocardial cushion matrix during embryonic heart development. Fibulin-2 is also synthesized by the smooth muscle precursor cells of developing aortic arch vessels and the coronary endothelial cells that originate from neural crest cells and epicardial cells, respectively (Tsuda et al., 2001).

The transcripts of the Hyaluronan Synthase gene (D84424), Fibromodulin gene (U0 5291), and the transcript 1NFLS (W03846) were not detected in USSCs, but are detected at high levels in MSCs. Northern blot analysis indicated that Hyaluronan Synthase is ubiquitously expressed in human tissues (Itano and Kimata, 1996). The product of this enzyme, Hyaluronan, serves a variety of functions, including space filling, lubrication of joints, and provision of a matrix through which cells can migrate (Hall et al., 1995). Fibromodulin is a member of a family of small interstitial proteoglycans. The protein exhibits a wide tissue distribution, with the highest abundance observed in articular cartilage, tendon, and ligament (Sztrolovics et al., 1994). The transcript 1NFLS was cloned from human fetal liver.

The CD24 gene (L33930) is expressed at a very low level in USSCs, compared with the expression level in MSCs. CD24 is expressed in many B-lineage cells and on mature granulocytes (Van der Schoot et al., 1989).

USSCs are characterized by no expression of human leukocyte antigen class I (HLA-class I). In contrast to USSCs, the previously described MSCs isolated from bone marrow and muscle tissue, express very high levels of HLA-class I antigen on their cell surface. USSCs also express the stage specific early antigen 4 (SSEA4).

Typically, USSCs show a fibroblastoid cell shape and proliferates in an adherent manner.

USSCs can be present in a plurality of mixtures representing precursors of other somatic stem cells, e.g. of the hematopoietic lineage expressing AC133 and CD34, mesenchymal progenitor somatic stem cells, neuronal progenitor somatic stem cells, or combinations thereof. Such combinations provide high regenerative potential based on the capability to differentiate into other, different somatic stem cells.

Some medicaments useful in the invention contain USSCs together with progenitor cells. The medicament may further contain carrier substances or auxiliary substances, which are medically and pharmacologically acceptable. USSCs and progenitor cells, when administered together, may be administered directly or with pharmaceutically acceptable carriers or adjuvants. It may be advantageous to add additional therapeutically active substances.

### Collection and Expansion of Umbilical Cord Blood Cells

The UCB cells can be collected by methods known in the art, see for example Koike et al., Acta Paediatrica Japonica 25:275-282, 1983, and expanded by methods described in, for example, U.S. Pat. No. 5,674,750, U.S. Pat. No. 5,925,567, and U.S. Pat. No. 6,338,942. Nucleated cells, e.g., progenitor cells, and adherent stroma cells, e.g., MSPCs, can be obtained from umbilical cord blood and can be used in accordance with the present invention. The nucleated cells can be expanded in the presence or the absence of adherent stroma cells under cell growth conditions, i.e., conditions that promote proliferation (“mitotic activity”) of the cells.

Isolation of Mononuclear Cells from Cord Blood

Umbilical cord blood was carefully loaded onto Ficoll a solution (density 1.077 g/cm³), and a density gradient centrifugation was performed (450 g, room temperature, 25 min.). The mononuclear cells (MNC) of the interphase were collected and washed twice in phosphate buffer saline, pH 7.3 (PBS).

Culture Conditions for the Expansion of Adherent Stroma Cells

Adherent stroma cells, such as MSPCs, can be expanded in H5100 medium containing 10 ng/ml insulin-like growth factor (IGF-I), 10 ng/ml platelet-derived growth factor (PDGF)-BB and 10 ng/ml recombinant human epidermal growth factor (rh-EGF) (PEI medium) at a density ranging from 1×10⁴ and 1×10⁵ cells/ml. Alternatively, adherent stroma cells preparations can be expanded in the initial growth medium.

Administration of Nucleated Cells Alone or in Combination with Adherent Stroma Cells

Various delivery systems are known and can be used to administer the nucleated cells, either as a co-culture in combination with adherent stroma cells, or separated from adherent stroma cells following co-culture. Methods of introduction include but are not limited to intradermal, intramuscular, intraperitoneal, intravenous, subcutaneous, intranasal, epidural, and oral routes. The cells may be administered by any convenient route, for example by infusion or bolus injection, and may be administered together with other biologically active agents. Administration can be systemic or local. In addition, it may be desirable to introduce the cells into the central nervous system by any suitable route, including intraventricular and intrathecal injection; intraventricular injection may be facilitated by an intraventricular catheter.

In a specific embodiment, it may be desirable to administer the cells of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as sialastic membranes, or fibers. In another embodiment, the cells or a cell preparation can be delivered in a vesicle, in particular a liposome (e.g., an encapsulated liposome).

Generally, methods known for the administration of MSCs can be applied in an analogous manner when administering adherent stroma cells. For example, the administration of stem cells is described in B. E. Strauer et al. M. “Intrakoronare, humane autologe Stammzelltransplantation zur Myokardregeneration nach Herzinfarkt”, Dtsch. Med. Wochenschr 2001; 126: 932-938; Quarto R., et al., “Repair of Large Bone Defects with the Use of Autologous Bone Marrow Stroma Cells”. N. Eng. J. Med. 2001; 344:385-386; Vacanti C. A., “Brief Report: Replacement of an Avulsed Phalanx with Tissue-Engineered Bone” N. Eng. J. Med. 2001; 344:1511-1514, May 17, 2001; Hentz V. R., “Tissue Engineering for Reconstruction of the Thumb”, N. Eng. J. Med. 2001; 344:1547-1548; Brittberg M., “Treatment of Deep Cartilage Defects in the Knee with Autologous Chondrocyte Transplantation”, N. Eng. J. Med 1994; 331:889-895, Oct. 6, 1994; Freed C. R., “Transplantation of a Tissue-Engineered Pulmonary Artery”, N. Eng. J. Med. 2001; 344:532-533. Shapiro A. M. J., Islet Transplantation in Seven Patients with Type 1 Diabetes Mellitus Using a Glucocorticoid-Free Immunosuppressive Regimen N. Eng. Med. 2000; 343:230-238. These references are hereby incorporated by reference.

### Systemic Infusions

Following co-culture of the nucleated cells and adherent stroma cells, the nucleated cells can be separated from the adherent stroma cells or they can remain together in the preparation and used immediately or stored for future use. If the cells are separated, the nucleated cells or adherent stroma cells can be further expanded, and, if so desired, used immediately thereafter or stored for future use. In instances where the number of cells in a single stored sample is insufficient, several such samples can be combined to provide the required number of cells. Further, rather than using the whole nucleated cell and/or adherent stroma cell population, sub-populations can be used that are enriched in a particular cell type, e.g., stem cells or other precursor cells (e.g., progenitor cells or MSPCs that have been expanded can be directed to differentiate into one or more pre-determined cell types and administered to the patient).

Following co-culture, the nucleated cells (either separated from or in combination with the adherent stroma cells) can be administered by infusion into the patient by, e.g., intracoronary infusion, retrograde venous infusion (see, e.g., Perin and Silva, Curr. Opin. Hematol. 11:399-403, 2004), intraventricular infusion, intracerebroventricular infusion, cerebrospinal infusion, and intracranial infusion.

It is anticipated that human therapy is likely to require multiple infisions of any cell composition prepared using the co-culture methods, as is discussed above, or various enriched and expanded sub-populations of the cells following co-culture. Several infusions of cells can be administered over time, e.g., one on day one, a second on day five, and a third on day ten. After the initial ten day period, there can be a period of time, e.g., two weeks to 6 months without cell administration, after which time the ten-day administration protocol can be repeated.

Whether administered as a single infusion therapy or multiple infusion therapies, it is likely that the recipient will require immunosuppression. The protocols followed for this will follow the precedents now used in human transplantation for bone marrow replacement (i.e., cell transplantation), with such agents as cyclosporin A and FK506.

Direct Injection

Another possible administration route for the nucleated cells alone or in combination with adherent stroma cells, or expanded sub-populations of these cells, is via direct surgical injection (e.g., intramyocardial or transendocardial injection, intracranial, intracerebral, or intracisternal injection, intramuscular injection, intrahepatic injection, and intrapancreatic injection) into the tissue or region of the body to be treated (e.g., the brain, muscle, heart, liver, pancreas, and vasculature). This method of administration may also require multiple injections with treatment interruption intervals lasting from 2 weeks to 6 months, or as otherwise determined by the attending physician.

Implantation

The nucleated cells and adherent stroma cells can also be administered by implantation into a patient at the site of disease or injury or at a site that will facilitate treatment of the disease or injury.

### Treatment of Disease

The method of the invention can be used to treat any human patients, whether children or adults, who suffer from one or more of the diseases or disorders disclosed, as is discussed below.

Nucleated cells alone or in combination with adherent stroma cells, once prepared as is described above to increase the cell numbers by co-culture, can be administered to a patient for the treatment of a disease or disorder. Preferably, the cells are typed for the patient using the standard six transplantation markers. While it is preferable that the cells exhibit a 6/6 match, the tolerizing effect of the adherent stroma cells (e.g., the MSPCs) on allogeneic cells, e.g., co-cultured nucleated cells from a donor other than the patient, allows the administration of cells that do not exhibit a 6/6 match, e.g., cells with less than a 4/6 match. Any rejection that does occur can be offset by using the standard methods described below, e.g., the administration of cyclosporin A or FK506.

Based on the beneficial effect of adherent stroma cells with respect to promoting engraftment of nucleated cells, the invention may be used to treat various diseases for which infusion and engraftment of such cells would aid in treating the disease. Such diseases may include without limitation leukemia, breast cancer, lymphoma, Hodgkin's disease, aplastic anemia, sickle cell anemia, various other cancers, blood diseases, hereditary/genetic conditions and immune system disorders, lung cancer, multiple sclerosis, lupus, AIDS, and many other genetic disorders.

In addition, the administered cells may be natural cells or may have engineered in them various genes that do not negatively alter the effectiveness of the cells in engrafting. The nucleated cells may be cultured with the adherent stroma cells before transplanting or they may be combined immediately prior to transplanting.

Organ/Tissue Regeneration

The progenitor cells of the invention or their progeny, either alone or in combination with adherent stroma cells or their progeny, can be used in a variety of applications. These include, but are not limited to, transplantation or implantation of the cells either in unattached form or as attached, for example, to a three-dimensional framework, as described herein. Typically, 10² to 10⁹ cells are transplanted in a single procedure, with additional transplants performed as required. The tissue produced according to the methods of the invention can be used to repair or replace damaged or destroyed tissue, to augment existing tissue, to introduce new or altered tissue, to modify artificial prostheses, or to join biological tissues or structures.

If the nucleated cells or adherent stroma cells are derived from a heterologous source relative to the recipient subject, concomitant immunosuppression therapy can be administered, e.g., administration of the immunosuppressive agent cyclosporine or FK506. However, due to the immature state of progenitor cells obtained from UCB and due to the tolerizing effect of the adherent stroma cells, e.g., MSPCs, on the nucleated cells, such immunosuppressive therapy may not be required. Accordingly, in one example, nucleated cells obtained from UCB can be administered, either alone or with adherent stroma cells and following co-culture of the cells or just after their combination, to a recipient in the absence of immunomodulatory (e.g., immunosuppressive) therapy.

In addition, injection of extracellular matrix prepared from new tissue produced by nucleated cells obtained from UCB, or their progeny, can be administered to a subject or may be used to further culture cells. Such cells, tissues, and extracellular matrix may serve to repair, replace or augment tissue that has been damaged due to disease or trauma, or that failed to develop normally, or for cosmetic purposes.

A preparation of nucleated cells obtained from UCB or their progeny and adherent stroma cells or their progeny can be injected or administered directly to the site where the production of new tissue is desired. For example, and not by way of limitation, the cells may be suspended in a hydrogel solution for injection. Alternatively, the hydrogel solution containing the cells may be allowed to harden, for instance in a mold (e.g., a vascular or tubular tissue construct), to form a matrix having cells dispersed therein prior to implantation. Once the matrix has hardened, the cell preparation may be cultured so that the cells are mitotically expanded prior to implantation. A hydrogel is an organic polymer (natural or synthetic) which is cross-linked via covalent, ionic, or hydrogen bonds to create a three-dimensional open-lattice structure, which entraps water molecules to form a gel. Examples of materials which can be used to form a hydrogel include polysaccharides such as alginate and salts thereof, polyphosphazines, and polyacrylates, which are cross-linked ionically, or block polymers such as PLURONICS™ or TETRONICS™ (BASF Corp., Mount Olive, N.Y.), polyethylene oxide-polypropylene glycol block copolymers which are cross-linked by temperature or pH. Methods of synthesis of the hydrogel materials, as well as methods for preparing such hydrogels, are known in the art.

Such cell preparations may further comprise one or more other components, including selected extracellular matrix components, such as one or more types of collagen known in the art, and/or growth factors and drugs. Growth factors which may be usefully incorporated into the cell formulation include one or more tissue growth factors known in the art or to be identified in the future, such as but not limited to any member of the TGF-β family, IGF-I and -II, growth hormone, BMPs such as BMP-13, and the like. Alternatively, nucleated cells and/or adherent stroma cells obtained from UCB may be genetically engineered to express and produce growth factors such as BMP-13 or TGF-β. Details on genetic engineering of the cells of the invention are provided herein. Drugs that may be usefully incorporated into the cell preparation include, for example, anti-inflammatory compounds, as well as local anesthetics. Other components that may also be included in the preparation include, for example, buffers to provide appropriate pH and isotonicity, lubricants, viscous materials to retain the cells at or near the site of administration, (e.g., alginates, agars, and plant gums) and other cell types that may produce a desired effect at the site of administration (e.g., enhancement or modification of the formation of tissue or its physicochemical characteristics, support for the viability of the cells, or inhibition of inflammation or rejection).

Nucleated cells and/or adherent stroma cells obtained from UCB can be administered directly, and those cells that are capable of differentiating can be induced to differentiate by contact with tissue in vivo or induced to differentiate into a desired or pre-determined cell type, e.g., mesenchymal cells, hematopoietic cells, neural cells, or endothelial cells, etc., using ill vitro or ex vivo methods before their administration. Such predisposition of progeny of progenitor cells and/or MSPCs obtained from UCB has the potential to shorten the time required for complete differentiation once the cells have been administered to the patient. Techniques for the differentiation of progenitor cells into cells of a particular phenotype are known in the art, such as those described in U.S. Pat. Nos. 5,486,359; 5,591,625; 5,736,396; 5,811,094; 5,827,740; 5,837,539; 5,908,782; 5,908,784; 5,942,225; 5,965,436; 6,010,696; 6,022,540; 6,087,113; 5,858,390; 5,804,446; 5,846,796; 5,654,186; 6,054,121; 5,827,735; and 5,906,934, which describe the transformation of pluripotent cells. For example, Rodgers et al. (U.S. Pat. No. 6,335,195), describes methods for the ex vivo culturing of hematopoietic and mesenchymal pluripotent cells and the induction of lineage-specific cell proliferation and differentiation by growth in the presence of angiotensinogen, angiotensin I (AI), AI analogues, AI fragments and analogues thereof, angiotensin II (AII), AII analogues, All fragments or analogues thereof, or AII AT₂-type 2 receptor agonists, either alone or in combination with other growth factors and cytokines. In an embodiment, the cell preparation can be administered to produce pancreatic cells, and in particular pancreatic islet cells, by using, e.g., techniques known in the art (see, e.g., Yang et al., Proc. Nat. Acad. Sci. USA 99: 8078-83, 2002; Zulewski et al., Diabetes 50: 521-33, 2001; and Bonner-Weir et al., Proc. Nat. Acad. Sci. USA 97: 7999-8004, 2001). Art-known techniques can also be used to promote the production of various other cell types upon administration of the co-cultured or co-transplanted cells of the invention, e.g., hepatic cells (see, e.g., Lee et al., Hepatology 40: 1275-1284, 2004), neuronal cells (see, e.g., Thondreau et al., Differentiation 319-322-326, 2004), or endothelial cells (see, e.g., Kassem et al., Basic Clin. Pharmacol. & Toxicol. 95:209-214, 2004; and Pittenger and Martin, Circ. Res. 95:9-20, 2004). Optionally, a differentiating agent may be co-administered or subsequently administered to the subject to promote stem cell differentiation in vivo.

The co-cultured or co-transplanted nucleated cells and adherent stroma cells, or their progeny, can be used to produce new tissue in vitro, which can then be implanted, transplanted, or otherwise inserted into a site requiring tissue repair, replacement, or augmentation in a subject. The co-cultured or co-transplanted nucleated cells and adherent stroma cells, or their progeny, may be inoculated or “seeded” onto a three-dimensional framework or scaffold, and proliferated or grown in vitro to form a living tissue which can be implanted in vivo. The three-dimensional framework may be of any material and/or shape that allows cells to attach to it (or can be modified to allow cells to attach to it) and allows cells to grow in more than one layer. A number of different materials may be used to form the matrix, including but not limited to: nylon (polyamides), dacron (polyesters), polystyrene, polypropylene, polyacrylates, polyvinyl compounds (e.g., polyvinylchloride), polyearbonate (PVC), polytetrafluorethylene (PTFE, teflon), thermanox (TPX), nitrocellulose, cotton, polyglycolic acid (PGA), collagen (in the form of sponges, braids, or woven threads, and the like), cat gut sutures, cellulose, gelatin, or other naturally occurring biodegradable materials or synthetic materials, including, for example, a variety of polyhydroxyalkanoates. Any of these materials may be woven into a mesh, for example, to form the three-dimensional framework or scaffold. The pores or spaces in the matrix can be adjusted by one of skill in the art to allow or prevent migration of cells into or through the matrix material. In one example, Naughton et al. U.S. Pat. No. 6,022,743), describe a tissue culture system in which stem cells or progenitor cells (e.g., cells such as those derived from umbilical cord cells, placental cells, mesenchymal stem cells or fetal cells) are propagated on three-dimensional supports.

The three-dimensional framework, matrix, hydrogel, and the like, can be molded into a form suitable for the tissue to be replaced or repaired. For example, where a vascular graft is desired, the three-dimensional framework can be molded in the shape of a tubular structure and seeded with the co-cultured or co-transplanted progenitor cells and USSCs, or their progeny. Other cells may also be added to the three-dimensional framework so as to improve the growth of, or alter, one or more characteristics of the new tissue formed thereon. Such cells may include, but are not limited to, fibroblasts, pericytes, macrophages, monocytes, plasma cells, mast cells, and adipocytes, among others.

Alternatively, the cells can be encapsulated in a device or microcapsule, which permits exchange of fluids but prevents cell/cell contact. Transplantation of microencapsulated cells is known in the art, e.g., Balladur et al., Surgery 117: 189-94, 1995; and Dixit et al., Cell Transplantation 1: 275-79, 1992. In one example, the cells may be contained in a device which is viably maintained outside the body as an extracorporeal device. Preferably, the device is connected to the blood circulation system such that the nucleated cells and adherent stroma cells can be functionally maintained outside of the body and serve to assist organ failure conditions. In another example, the encapsulated cells may be placed within a specific body compartment such that they remain functional for extended periods of time in the absence or presence of immunosuppressive or immuno-modulatory drugs.

In yet another example, the co-cultured or co-transplanted nucleated cells and adherent stroma cells, or their progeny, can be used in conjunction with a three-dimensional culture system in a “bioreactor” to produce tissue constructs which possess critical biochemical, physical and structural properties of native human tissue by culturing the cells and resulting tissue under environmental conditions which are typically experienced by the native tissue. Thus, the three-dimensional culture system may be maintained under intermittent and periodic pressurization and the cells of the invention provided with an adequate supply of nutrients by convection. Maintaining an adequate supply of nutrients to the cells of the invention throughout a replacement endothelial tissue construct of approximately 2-5 mm thickness is important as the apparent density of the construct increases. Pressure facilitates flow of fluid through the three-dimensional endothelial construct, thereby improving the supply of nutrients and removal of waste from cells embedded in the construct. The bioreactor may include a number of designs. Typically the culture conditions will include placing a physiological stress on the construct containing cells similar to what will be encountered in vivo. For example, the vascular construct may be cultured under conditions that simulate the pressures and shear forces of blood vessels (see, for example, U.S. Pat. No. 6,121,042, which is hereby incorporated by reference herein).

The methods of the invention may be used to treat subjects requiring the repair or replacement of tissue, e.g., endothelial tissue, resulting from disease or trauma, or to provide a cosmetic function, such as to augment facial or other features of the body. Treatment may entail the in vivo use of the nucleated cells and adherent stroma cells, or their progeny, to produce new tissue, or the use of the tissue produced in vitro or ex vivo, according to any method presently known in the art or to be developed in the future.

In another example, the nucleated cells and adherent stroma cells prepared according to the methods of the invention are administered to repair or replace a heart valve, vascular tissue, or graft. In another example, the nucleated cells and adherent stroma cells are administered in combination with angiogenic factors to induce or promote new capillary or vessel formation in a subject. By “angiogenic factor” is meant a growth factor, protein or agent that promotes or induces angiogenesis in a subject. The cells of the invention can be administered prior to, concurrently with, or following injection of the angiogenic factor. In addition, the nucleated cells and adherent stroma cells may be administered immediately adjacent to, at the same site, or remotely from the site of administration of the angiogenic factor.

As cardiac muscle does not normally have reparative potential, the nucleated cells and adherent stroma cells or their progeny can be used to regenerate or repair striated cardiac muscle that has been damaged through disease or degeneration. In such a therapy, the nucleated cells and adherent stroma cells are administered to the patient integrate with the healthy tissue of the recipient and replace the function of the dead or damaged cells, thereby regenerating the cardiac muscle as a whole. The nucleated cells and adherent stroma cells are used, for example, in cardiac muscle regeneration for a number of principal indications: (i) ischemic heart implantations, (ii) therapy for congestive heart failure patients, (iii) prevention of further disease in patients undergoing coronary artery bypass graft, (iv) conductive tissue regeneration, (v) vessel smooth muscle regeneration, and (vi) valve regeneration.

The nucleated cells and adherent stroma cells used in the invention, or their progeny, can be useful in the treatment of pancreatic or hepatic diseases or disorders. For example, cells may be implanted, injected, or otherwise administered directly to the site of damage so that they will produce new pancreatic or hepatic tissue in vivo. Methods of treatment include identifying a patient having a extraintestinal gastrointestinal or a hepaticopancreatic disorder and administering to the patient a therapeutically effective amount of a preparation that includes the cells, or their progeny, to treat the disorder. An “extraintestinal gastrointestinal” disorder is a disorder of the gastrointestinal tract that is primarily localized in an area other than the interior of the intestine. Non-limiting examples of extraintestinal gastrointestinal disorders include hepaticopancreatic disorders, duodenum disorders, bile duct disorders, appendix disorders, spleen disorders, and stomach disorders. “Hepaticopancreatic” disorders are disorders of the pancreas and liver. Non-limiting examples of hepaticopancreatic disorders include diabetes, pancreatitis, hepatic cirrhosis, hepatitis, cancer and pancreatico-biliary disease. A “disorder” of a particular organ or structure includes situations where the organ or structure is entirely absent. For example, for the purposes of this invention, a person who lacks a pancreas has a pancreatic disorder. Methods of implanting exogenous tissue are well known (see, e.g., J. Shapiro et. al., New Engl. J. Med. 343: 230-238, 2000, for the transplantation of pancreatic islets).

The nucleated cells and adherent stroma cells of the invention, or their progeny, can be useful in the treatment of neural diseases. In one example, the cells are administered to a patient to promote neurogenesis or gliogenesis in the central nervous system, such as the brain. Such treatment may be aimed at patients with Parkinson's disease, Alzheimer's disease, or who have suffered a stroke or trauma. In the case of glial cells, the therapy may be intended for treating multiple sclerosis and other glia related conditions. Other examples of tissues that could be generated are the optic stalk, retinal layer, and lens of the eye, and the inner ear. In certain methods, the patient may have suffered a neurodegenerative disease, a traumatic injury, a neurotoxic injury, ischemia, a developmental disorder, a disorder affecting vision, an injury or disease of the spinal cord, or a demyelinating disease. These patients having a neural disease or disorder that may be associated with impaired function can be administered a pharmaceutically effective amount of the cell preparation such that neurons, or other beneficial cell types, are produced depending on the neural disease or disorder to be treated.

Immune Reconstitution and Serial Injections using Nucleated Cells and Adherent Stroma Cells

Nucleated cells co-cultured and/or cotransplanted with adherent stroma cells according to the methods disclosed herein can be administered to reconstitute the immune system of a patient in need thereof, e.g., a patient who has undergone ablation of bone marrow during therapy. If the recipient's own cells are not available, donor cells may be used. The donor nucleated cells and adherent stroma cells that reconstitute the ablated marrow will effectively tolerize to the host and thereby survive after a period of weeks or months without on-going immunosuppression. The nucleated cells and adherent stroma cells used in the invention, or their progeny, can be administered once or serially to reconstitute the immune system of the patient.

Maintenance of the donor nucleated cells and adherent stroma cells within a host may require chronic use of one or more immunosuppressant regimens. This is analogous to the long-term use of immunosuppressant drugs or other therapy in patients who undergo whole organ or cell transplantation. Although, as is discussed above, the tolerizing effect of the adherent stroma cells on the nucleated cells may make the use of immunosuppressant drugs unnecessary. The necessity of administering an immunosuppressant drug can be evaluated by the patient's physician and, if necessary, immunosuppressive therapy can be initiated.

### Differentiation

After the nucleated cells and adherent stroma cells used in the invention have been isolated and expanded according to the methods of the invention, those progenitor cells present in the co-culture can be maintained for a substantial length of time in an undifferentiated state (e.g., 2 to 4 hours, 1 to 5 days, 1 to 14 days, 1 to 6 months, or indefinitely). It is desirable that substantially no differentiation of the cells occur during expansion. The amount of differentiation that occurs can be determined using assays known to one skilled in the art, e.g., those that detect the presence of more differentiated cells by detecting functions associated with a particular stage of differentiation, e.g., expression of differentiation antigens on the cell surface or secretion of proteins associated with a particular state, or the ability to generate various cell types; or by detecting changes in the morphology of the cells that are known to be associated with differentiation of the cells (see, WO 00/34443 for assays that test the differentiation/functional characteristics of HSCs).

Once the cells have been expanded to the desired numbers, they can be administered in an undifferentiated state, or the cells can be administered following induction of the cells to differentiate to a particular cell type. Optionally, the cells can be differentiated to a terminally differentiated state if the function of that terminally differentiated cell is desired.

### Gene Therapy

Gene therapy can also be used to modify the nucleated cells and adherent stroma cells to provide one or more missing protein(s) that are the basis for a disease or disorder. It is envisioned that the nucleated cells and adherent stroma cells can be used to replace bone marrow cells or can be modified with a corrected gene product and administered to the patient using one or more of the methods described above to treat or prevent a disease or disorder. Therapeutic uses of the UCB-derived cells that have been genetically transformed include transplanting the cells, cell populations, or progeny thereof into individuals to treat a variety of pathological states including diseases and disorders resulting from myocardial damage, circulatory or vascular disorders or diseases, neural diseases or disorders, hepatic diseases or disorders, or pancreatic diseases or disorders, as well as tissue regeneration and repair. By the same techniques described above, the genetically altered cells, or progeny thereof, used in the methods of the invention can be administered to a subject in need of such cells or in need of the protein or molecule encoded or produced by the genetically altered cell. Delivery of the altered cells would then treat the patient's disease or disorder.

An exemplary therapeutic gene therapy regimen may include the steps of obtaining nucleated cells and adherent stroma cells from the UCB of a subject or donor, enrichment in vitro, expansion of the nucleated cells by methods known in the art or by co-culture with adherent stroma cells, transduction of the nucleated cells and/or adherent stroma cells with a vector containing a gene of interest, and reintroduction into the subject. Transduction of the nucleated cells and/or adherent stroma cells by gene therapy techniques can be during or after expansion.

For example, genes that express products capable of preventing or ameliorating symptoms of various types of diseases or disorders (e.g., vascular diseases or disorders) or that prevent or promote inflammatory disorders can be incorporated into nucleated cells and adherent stroma cells obtained from UCB. In one example, these cells are genetically engineered to express an anti-inflammatory gene product that would serve to reduce the risk of failure of implantation or further degenerative change in tissue due to inflammatory reaction. The expression of one or more anti-inflammatory gene products include, for example, peptides or polypeptides corresponding to the idiotype of antibodies that neutralize granulocyte-macrophage colony stimulating factor (GM-CSF), TNF-α; IL-1, IL-2, or other inflammatory cytokines. IL-1 has been shown to decrease the synthesis of proteoglycans and collagens type II, IX, and XI (Tyler et al., Biochem. J. 227: 69-878, 1985; Tyler et al., Coll. Relat. Res. 82: 393-405, 1988; Goldring et al., J. Clin. Invest. 82: 2026-2037, 1988; and Lefebvre et al., Biophys. Acta. 1052: 366-72, 1990). TNF-α also inhibits synthesis of proteoglycans and type II collagen, although it is much less potent than IL-1 (Yaron et al., Arthritis Rheum. 32: 173-80, 1989; Ikebe et al., J. Immunol. 140: 827-31, 1988; and Saklatvala Nature 322: 547-49, 1986). Also, for example, progenitor cells and/or MSPCs obtained from UCB may be engineered to express the gene encoding the human complement regulatory protein that prevents rejection of a graft by the host. See, for example, McCurry et al., Nature Medicine 1: 423-27, 1995. In another example, nucleated cells and adherent stroma cells obtained from UCB can be engineered to include a gene or polynucleotides sequence that expresses or causes to be expressed an angiogenic factor.

Alternatively, nucleated cells and adherent stroma cells obtained from UCB may be genetically engineered to express and produce growth factors such as VEGF, FGF, EGF, IGF, as well as therapeutic agents such as TWEAK, TWEAKR, TNFR, other anti-inflammatory agents, or angiogenic agents. For example, the gene or coding sequence for such growth factors or therapeutic agents would be placed in operative association with a regulated promoter so that production of the growth factor or agent in culture can be controlled.

In another example, nucleated cells and adherent stroma cells obtained from UCB are genetically modified or engineered to contain genes which express proteins of importance for the differentiation and/or maintenance of striated cardiac muscle cells. Examples include growth factors (TGF-α, IGF-1, FGF), myogenic factors (myoD, myogenin, Myf5, MRF), transcription factors (GATA-4), cytokines (cardiotrophin-1), members of the neuregulin family (neuregulin 1, 2 and 3) and homeobox genes (Csx, tinman, NKx family).

Alternatively, the nucleated cells and adherent stroma cells may be genetically engineered to “knock out” expression of native gene products that promote inflammation, e.g., GM-CSF, TNF, IL-1, IL-2, or “knock out” expression of MHC in order to lower the risk of rejection. In addition, the cells may be genetically engineered for use in gene therapy to adjust the level of gene activity in a subject to assist or improve the results of transplantation.

Genetically engineered nucleated cells and adherent stroma cells may also be screened to select those cell lines that bring about the amelioration of symptoms of rheumatoid disease or inflammatory reactions in vivo, and/or escape immunological surveillance and rejection.

Several methods are known in the art for altering cells for use in gene therapy. These methods include cell transduction (see, e.g., Beutler, Biol. Blood Marrow Transplant 5:273-276, 1999; Dao, Leukemia 13:1473-1480, 1999; and see generally Morgan et al., Ann. Rev. Biochem. 62:191-217, 1993; Culver et al., Trends Genet. 10:174-178, 1994; and U.S. Pat. No. 5,399,346 (French et al.)); the use of viral vectors; and the use of non-viral vectors, for example, naked DNA delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, and pressure-mediated gene delivery.

The technique should provide for the stable transfer of the gene to the cell, so that the gene is expressible by the cell and preferably heritable and expressible by its cell progeny.

For general reviews of the methods of gene therapy, see Goldspiel et al., Clinical Pharmacy 12:488-505, 1993; Wu and Wu, Biotherapy 3:87-95, 1991; Tolstoshev, Ann. Rev: Pharmacol. Toxicol. 32:573-596, 1993; Mulligan, Science 260:926-932, 1993; and Morgan and Anderson, Ann. Rev. Biochem. 62:191-217, 1993. Methods commonly known in the art of recombinant DNA technology that can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The invention is further described in the following non-limiting examples.

### EXAMPLE 1

Provided herein is the examined the immunophenotype, the supporting function in relation to ex vivo expansion of progenitor cells (e.g., CD34⁺, Lin⁻ cells), and the chondrogenic differentiation of cultured cells with characteristics of MSPCs from UCB. UCB nucleated cells were isolated and 10⁷ cells were cultured in IMDM with 20% fetal bovine serum. The mean number of adherent fibroblastlike colonies was 3.5±0.7/10⁶ monuclear cells.

UCB-derived MSPCs could be expanded for at least 15 passages. In their undifferentiated state, UCB-derived MSPCs were CD13⁺, CD29⁺, CD9⁺, CD105⁺, CD166⁺, SH2⁺, SH3⁺, SH4⁺, CD45⁻, CD34⁻, and CD14⁻; they produced stem cell factor, interleukin 6 and tumor necrosis factor a UCB-derived MSPCs cultured in chondrogenic media differentiated into chondrogenic cells. UCB-derived MSPCs supported the proliferation and differentiation of CD34⁺ cells from UCB in vitro.

UCB-derived MSPCs have the potential to support ex vivo expansion of progenitor cells (e.g., CD34⁺, Lin⁻ cells) and chondrogenic differentiation. UCB should not be regarded as medical waste. It can serve as an alternative source of progenitor stem cells and supporting cells, and may provide a unique source of fetal cells for cellular and gene therapy.

### Umbilical Cord Blood (UCB)

UCB, collected for research purposes, was kindly provided by the Obstetrics Department of the Zhejiang Gynecological and Obstetric Hospital, Hangzhou. Twenty-four parturient women with a median age of 27 years (range 25 to 30 years) and a median weight of 60 kg (range 52 to 68 kg), gave written consent to the use of UCB for research purposes in accordance with procedures approved by the Human Experimentation Committee at Zhejiang Public Health Bureau, Hangzhou, P. R. China The collected UCB was heparinized.

### Isolation and Culture of UCB-Derived MSPCs

A median of 72 mL of UCB (range 60 to 83 mL) was centrifuged at 450×g for 10 min within 12 hours after collection. The pellet was diluted with Iscove's modified Dulbecco's medium (IMDM; HyClone, Logan, Utah) and then layered onto Ficoll-Hypaque (1.077±0.001 g/mL; Sigma, St. Louis, Mo., USA), and centrifuged at 300×g for 20 min. Low-density mononuclear cells (MNC) from the gradient interface were collected and washed three times with IMDM and were then diluted with the complete medium (20% fetal bovine serum (FBS; Sigma) in IMDM with 50 μM 2 mercaptoethanol and 2 mM L-glutamine (Gibco BRL, Life Technologies, Paisley, UK). The resuspended cells were cultured in 25-cm² flasks at a density of 4.2×10⁵ cells/cm², and incubated in 100% humidified 5% CO² in air at 37° C. After 4 days, when cells had adhered to the flask, the supernatant and non-adherent cells were removed, and the complete medium was replaced. At 90% confluence, cells were harvested with 0.25% trypsin and 1 mM EDTA (Stem Cells Technology, Vancouver, BC, Canada) for 5 minutes at 37° C. Harvested cells were washed twice with PBS/1%FBS and then CD34 negative cells were isolated using a MACS laboratory separation system (Miltenyi Biotec, Bergisch Gladbach, Germany) according to the manufacturer's instructions. The CD34⁻ cells were diluted with complete media and cultured in 25-cm² flasks at a density of 5×10³ cells/cm², in 100% humidified 5% CO² in air at 37° C. as the F₁ passage. At near confluence, cells were harvested and cultured by a passage as described above. In order to measure the growth kinetics, cells from passages F₁, F₅, F₁₀ and F₁₅ were plated into a 6-well plate at 10⁴ cells/cm². Growth curves were assessed once every four days, by counting the number of adherent cells, for 24 days.

### Flow Cytometry Analysis of Cultured UCB-Derived MSPCs

UCB-derived adherent cells (at the end of passages F₁, F₅, F₁₀ and F₁₅) were trypsinized and stained with anti-CD34-FITC, CD45-FITC, CD14-FITC, CD105-FITC, CD90-PE, CD166-PE, CD13-PE, CD29-PE (Becton Dickinson, UK), SH2, SH3, and SH4 monoclonal antibodies (Osiris Therapeutics, Baltimore, Md., USA) and were analyzed by FACScalibur flow cytometry (Becton Dickinson).

### Cytokine Production in the Conditioned Medium of Cultured UCB-Derived MSPCs

When adherent cells in passages F₁, F₅, and F₁₀ were confluent, the conditioned media were collected and used to measure any cytokines produced by the UCB-derived adherent cells. The presence of stem cell factor (SCF), granulocyte-macrophage colony stimulating factor (GM-CSF), interleukin 3 (IL-3), interleukin 6 (IL-6) and tumor necrosis factor a (TNF-α) in the media was quantitatively determined by enzyme-linked immunosorbent assay (ELISA) kits (R&D Systems, Minneapolis, Minn., USA) following the manufacturer's instructions. The limit of detection of each ELISA kit was 4.0 pg/mL for SCF, 7.8 pg/mL for IL-3, 20 pg/mL for GM-CSF, 0.09 pg/mL for IL-6, and 0.18 pg/mL for TNF-α.

### Co-Culture of UCB-Derived MSPCs and CD34⁺ Hematopoietic Cells

The UCB-derived adherent cell layer from the F₅ passage was selected for the experiments investigating the support offered to ex vivo expansion of HSPC. This passage was chosen in consideration of the homogeneous state of cells, their growth potential and cytokine production. MNC from UCB were isolated as above. CD34⁺ cells, selected from MNC preparations with anti-CD34 antibodies (Miltenyi Biotec) conjugated with microbeads and eluted through MiniMACS columns according to the manufacturer's instructions, were resuspended in IMDM (20% FBS) with or without 100 ng/mL each of recombinant human stem cell factor (rhSCF), recombinant human granulocyte colony-stimulating factor (rhG-CSF) and recombinant human megakaryocyte growth and development factor (rhMGDF) (Amgen Inc., Thousand Oaks, Calif., USA), and then seeded at the density of 1.6×10⁴ cells/cm² in 25-cm² flasks with the UCB-derived adherent MSPCs. As a control system, CD34⁺ cells were cultured in IMDM (20% FBS) with the same concentration of three exogenous cytokines but without the UCB-derived adherent MSPCs. The UCB-derived CD34⁺ cells were cultured in 100% humidified 5% CO² in air at 37° C. using a two-step culture system as previously described (McNiece et al., Exp. Hematol. 28:1181-1186, 2000). At the beginning (the first day) and at the end (the 14th day) of culture, non-adherent cells in the culture medium were assayed for colony-forming cells in complete methylcellulose without erythropoietin (Gencyte, Amherst, N.Y., USA) as the progenitor cell assay.

### Chondrogenic Differentiation and Analysis of Collagen Gene Expression

The differentiating ability of MSPCs was assessed at passages F₁ , F₅ and F₁₀ of UCB-derived MSPCs (n=5). Chondrogenic medium containing DMEM supplemented with 10% FBS, 10 ng/mL transforming growth factor-β1 (TGF-β1) (R&D Systems), 50 μg/mL ascorbic acid, 100 U/mL penicillin and 100 μg/mL streptomycin was used to induce chondrogenic differentiation. After 3 weeks of induction, the induced cells were fixed with 10% formalin solution for 1 h, washed once with distilled water, and then stained in 1% toluidine blue solution for 2-3 h. Total RNA was prepared by the guanidinium thiocyanate/phenol method. Reverse transcription was performed by denaturing RNA and dT18 primers in the presence of 0.1 mol/L methylmercuric hydroxide, followed by quenching with 20 mmol/L β-mercaptoethanol and extension in a total of 20 μL with Superscript II reverse transcriptase as recommended (GIBCO, Carlsbad, Calif., USA). Polymerase chain reactions (PCR) were performed using 2.0 μL RNase-treated cDNA with Taq polymerase (Perkin Elmer, Foster City, Calif., USA) in a total of 50 μL. The PCR reactions were performed with an initial denaturation of 94° C. for 2.0 minutes, and then at 94° C. for 0.5 minutes, 58° C. for 0.75 minutes, and 68° C. for 0.75 minutes for 10 cycles, followed by 94° C. for 0.5 minutes, 58° C. for 0.75 minutes, and 73° C. for 0.75 minutes for another 25 cycles. The primer set used for PCR of collagen cDNA was as follows: 5′ TTC AGC TAT GGA GAT GAC AAT C 3′ and 5′ AGA GTC CTA GAG TGA CTG AG 3′. Fifteen microliters of PCR reaction were fractionated by agarose gel electrophoresis.

### Statistics

Results are expressed as mean ± SEM, and statistical comparisons were performed using the Student's t test.

### Adhesion, Fibroblastic and Growth Characteristics of UCB-Derived MSPCs

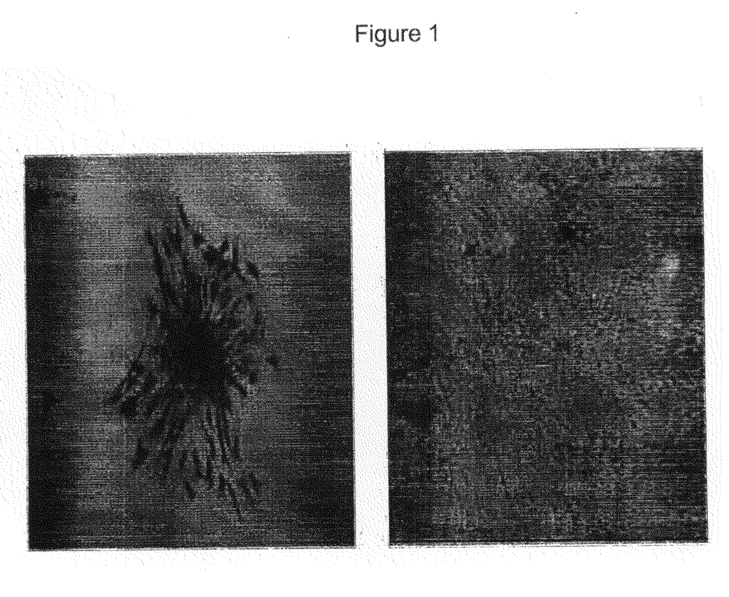

Cells adhered to flasks and formed individual colonies consisting of several dozen to a few hundred spindle-shaped fibroblastic cells by day 7-9 after UCB MNC had been inoculated at a density of 4.2×10⁵ cells/cm² in IMDM containing only 20% FBS with no additional growth factors (FIG. 1A). The frequency of adherent colonies was 3.5±0.7/10⁶ MNC. After 15-19 days of culture, numerous fibroblast-like cells could be observed. By the end of four weeks, a homogeneous layer of fibroblastoid cells occupied the whole surface of the flask and had become confluent (FIG. 1B).

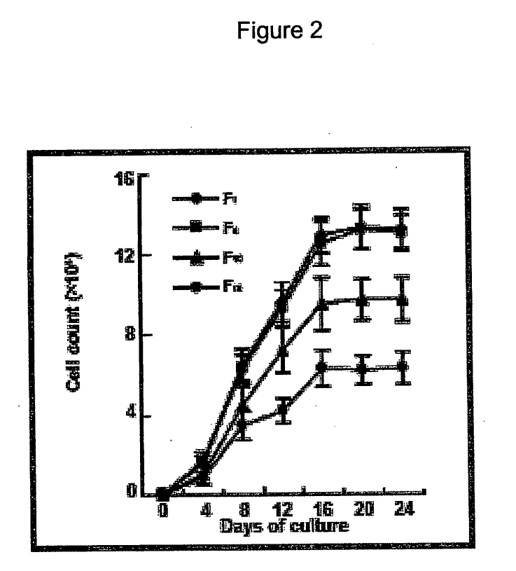

The growth kinetics of UCB-derived adherent cells (n=5) was measured at passages F₁, F₅, F₁₀ and F₁₅ (FIG. 2). Cells were allowed to divide for 24 days, duplicate cultures were harvested once every four days, and cell counts were performed. Growth curves depicted an initial lag phase of 4 days, followed by a log phase in which cells divided at an exponential rate for 8 to 12 days. The log phase was followed by a plateau phase. UCB-derived adherent cells could be readily expanded in vitro by successive cycles of trypsinization, seeding, and culture every 17 to 20 days for 15 passages. Cells that had undergone up to 15 passages displayed no visible change in their morphology, their forward and side scatter properties, or their growth patterns, but fold expansion decreased from F₁₀ to F₁₅.

To assess the capacity of UCB-derived adherent cells to secrete hematopoietic cytokines, conditioned medium was sampled from the F₁, confluent passage. SCF, IL-6, and TNF-α levels were detected in conditioned media while IL-3 and GM-CSF were not detected (Table 1).

TABLE 1
Cytokine production in the conditioned media of cultured
UCB-derived mesenchymal stem/progenitor cells (pg/mL).

| | SCF | IL-3 | IL-6 | GM-CSF | TNF-a |
|---|---|---|---|---|---|
| F₁ | 129.6 ± 11.2* | — | 75.2 ± 9.8* | — | 57.4 ± 7.7* |
| F₅ | 34.2 ± 9.7 | — | 78.2 ± 11.2 | — | 61.2 ± 9.7 |
| F₁₀ | 32.1 ± 12.4 | — | 77.4 ± 13.1 | — | 62.2 ± 7.8 |

F₁, F₅, and F₁₀ are F₁passage, F₅passage and F₁₀passage, respectively. SCF: stem cell factor; IL-3: interleukin 3; IL-6: interleukin 6; GM-CSF: granulocyte-macrophage colony-stimulating facto; TNF-α: tumor necrosis factor α.
Comparison with F₅passage:
*p < 0.05.
—: undetected. 2₃

### Immunophenotype of UCB-Derived MSPCs

The immunophenotype of UCB-derived adherent cells from passages F₁, F₅, F₁₀ and F₁₅ was determined by flow cytometry. As shown in Table 2, UCB-derived monolayer adherent cells stained positively for CD13, CD29, CD90, CD105, CD166, SH2, SH3 and SH4. CD34⁺ cells were not detected in UCB adherent cells from any passage because this cell population had been depleted at the beginning of passage F₁. Low levels of CD14 and CD45 were detected in F₁ probably due to contaminating cells, but no significant staining was seen at F₅ and F₁₀. This profile is consistent with a non-hematopoietic cell and confinned that hematopoietic cells had been depleted from the cultures. The immunophenotypic profile of UCB adherent cells did not change significantly after 10 passages in culture p>0.05).

TABLE 2
Percentage of UCB-derived monolayer adherent cells expressing specific phenotypes (%).

| | CD13 | CD14 | CD29 | CD45 | CD90 | CD105 | CD166 | SH2 | SH3 | SH4 |
|---|---|---|---|---|---|---|---|---|---|---|
| F₅ | 1.8 ± 8.7° | 3.6 ± 2.3* | 48.8 ± 7.8° | 8.4 ± 2.1° | 62.4 ± 10.5° | 45.2 ± 15.2° | 68.8 ± 9.3° | 45.2 ± 4.9° | 53.0 ± 9.5° | 39.8 ± 10.4° |
| F₅ | 76.4 ± 5.2* | 1.8 ± 1.7 | 81.2 ± 14.2* | 3.2 ± 1.0* | 87.2 ± 12.7 | 58.0 ± 11.3* | 91.6 ± 18.1 | 61.8 ± 7.2* | 77.6 ± 5.4 | 63.8 ± 11.2* |
| F₁₀ | 83.2 ± 12.4 | 0.5 ± 0.1 | 87.4 ± 9.5 | 1.2 ± 0.8 | 82.8 ± 8.8 | 64.2 ± 6.9 | 93.2 ± 12.7 | 77.4 ± 12.5 | 82.8 ± 7.6 | 74.4 ± 8.3 |
| F₁₅ | 82.7 ± 9.2 | 0.6 ± 0.2 | 88.6 ± 11.3 | 1.0 ± 0.9 | 83.1 ± 10.7 | 66.0 ± 8.8 | 93.1 ± 11.2 | 78.4 ± 11.3 | 83.1 ± 9.2 | 75.8 ± 9.9 |

F₁, F₅, F₁₀ and F₁₅ are the F₁passage, F₅passage, F₁₀passage and F₁₅passage, respectively.
Comparison with F₁₀passage:
*p < 0.05,
°p < 0.01.

### Ability of UCB-Derived MSPCs to Support Ex Vivo Expansion of Progeinitor Cells

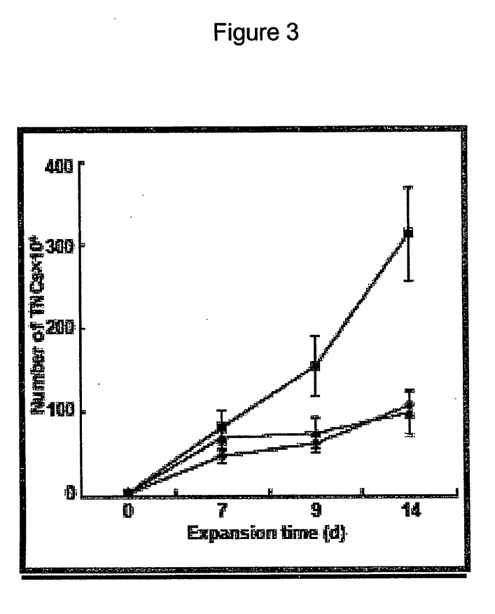

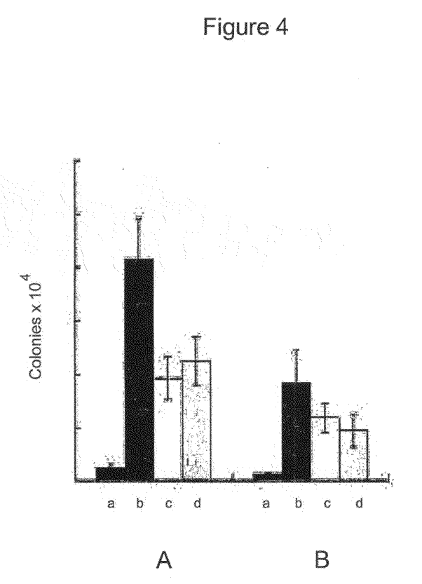

To determine whether MSPCs from UCB were capable of supporting the ex vivo proliferation and differentiation of progenitor cells (e.g., CD34⁻, CD34⁺, Lin⁻ HSPC), confluent monolayer MSPCs of passage F₅ (n=5) were used to co-culture human CD34⁺ cord blood cells. CD34⁺ cells were overlaid and maintained in culture for up to 14 days. As controls, UCB CD34⁺ cells were seeded into the 25-cm² flask with the same medium but without UCB-derived USSCs as a stroma cell feeder layer. FIG. 3 shows the growth kinetics of the non-adherent cells in the three culture systems. The expansion of total nucleated cells (TNC) in the co-culture system with exogenous cytokines was higher than that in the co-culture system without exogenous cytokines and in the control system (p<0.01). The number of expanded TNC in the co-culture system with exogenous cytokines was 3.16 (±0.3)-fold higher than that in the control system. The level of progenitor cells was assayed from non-adherent cells in the culture medium at the beginning (the first day) and at the end (the 14th day) of culture. As shown in FIG. 4, MSPCs from UCB increased the expansion of GM-CFC and HPP-CFC (p<0.05). The mean number of GM-CFC and HPP-CFC in the starting CD34⁺ cell fraction was 2.3 (±0.6)×10⁴ and 1.4 (±0.3)×10⁴, respectively. After expansion, there were 41.8 (±7.5)±10⁴ GM-CFC and 18.4 (±6.1)×10⁴ HPP-CFC for the co-culture system with exogenous cytokines, and 22.5 (±4.5)×10⁴ GM-CFC and 9.5 (±3.1)×10⁴ HPP-CFC for the control system.

The percentage of CD34⁺ cells decreased after 14 days of expansion (5.7%, 4.8% and 2.9%, respectively, for the co-culture system with exogenous cytokines, the co-culture system without exogenous cytokines and the control system) compared to the starting percentage (97.1% for CD34⁺ cell fraction). However, the co-culture conditions resulted in 6.2-fold higher numbers of CD34⁺cells than those grown in the control culture.

Differentiation of UCB-Derived MSPCs into Chondrocytes

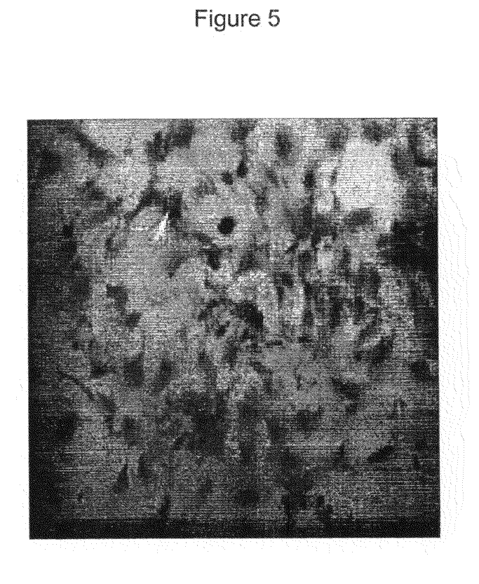

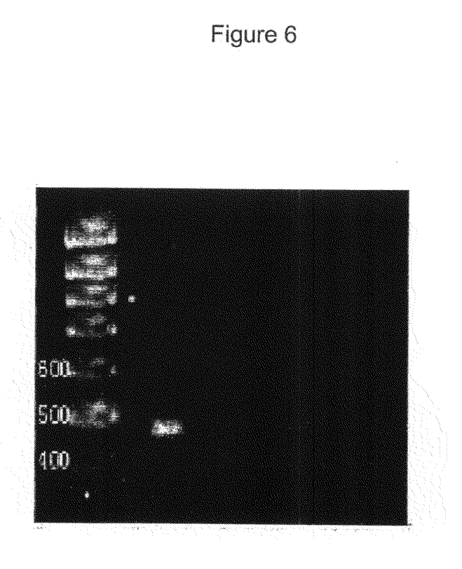

The chondrocytic differentiation potential of UCB-derived MSPCs in passages F₁, F₅ and F₁₀ was studied by culturing cells under conditions suitable for inducing chondrogenic differentiation. After 3 weeks of culture in chondrogenic medium, induced cells formed chondrocyte-like lacunae which were visualized by light microscopy; furthermore, the induced cells secreted a metachromatic matrix, which was positive with toluidine blue staining (FIG. 5). To confirm this differentiation, we used RT-PCR to analyze collagen gene expression. Total RNA from differentiated chondrocyte-like cells and undifferentiated UCB USSCs was prepared and CDNA of total mRNA was synthesized by reverse transcription. A fragment of approximately 500 bp was amplified from differentiated chondrocyte-like cells by RT-PCR with a pair of primers specific for the collagen gene (FIG. 6). By contrast, no amplified products were found from undifferentiated MSPCs. This chondrocytic differentiation capacity was retained for passages F₅ and F₁₀.

It has been demonstrated herein that there is a higher CD34⁺ CD38⁻ cell fraction in UCB than in normal adult bone marrow, suggesting that very primitive progenitor cells may be more abundant in UCB. Transplantation studies with UCB products have been shown to provide long-term durable engraftment in vivo, but the time to achieve neutrophil and platelet engraftment is longer in UCB recipients than in recipients of bone marrow and peripheral blood products. These and many other studies are consistent with UCB containing higher levels of primitive stem cells. Other studies have demonstrated that human UCB can be routinely cultured to form a confluent adherent feeder layer. It is well known that adult bone marrow-derived MSC can be rapidly expanded, differentiate into multiple terminal cells, and support expansion of hematopoietic stem cells in vitro. MSPCs from UCB also have these characteristics and functional properties such as supporting proliferation of hematopoietic stem cells, and differentiating into osteogenic, adipogenic, or neurogenic cells. In this study we demonstrated that UCB-derived MSPCs can also be differentiated into chondrogenic cells.

In adult bone marrow, mesenchymal cells provide signals for differentiation and proliferation of hematopoietic stem cells and their progeny through direct cell-cell interactions and secretion of hematopoietic growth factors and cytokines. It has also been demonstrated that proliferation and differentiation of progenitor cells occurs in a number of histologically distinct microenvironments (yolk sac, ventral aorta, fetal liver, thymus, spleen, and bone marrow) during human ontogeny. Shown herein is that USSCs from UCB support the proliferation and differentiation of progenitor cells from the same tissue, but the relationship between MSPCs and progenitor cells in human UCB during ontogeny remains to be determined. Hematopoietic growth factors and cytokines play a critical role in the proliferation and differentiation of HSPC. ELISA assays were used to screen for SCF, IL-3, IL-6 GM-CSF and TNF-A production. Only SCF, IL-6 and TNF-A were detected in the conditioned medium of UCB adherent cell layer cultures. There is some controversy about GM-CSF secretion by human bone marrow derived-adherent cells. Ye et al. also failed to detect endogenous IL-3 and GM-CSF in the conditioned medium of UCB-derived adherent cell layer cultures. We should, however, note the TNF-α detected in the conditioned media. This may induce cells to express mRNA of many hematopoietic factors, such as IL-1, IL-6, IL-8 and GM-CSF. Coordinated regulation of various growth factors produced or induced by human UCB-derived adherent cells in co-culture may support ex vivo proliferation and differentiation of human UCB-derived progenitor cells. Although other cytokines in the non-co-culture system (such as IL-6 and TNF-α) were not examined in the co-culture systems, ex vivo expansion of progenitor cells in co-culture with exogenous cytokines was, indeed, greater than that in the non-co-culture. A co-culture experiment without exogenous cytolines also showed that UCB-derived adherent cells supported the expansion of progenitor cells. Perhaps other hematopoietic growth factors and cytokines that were undetected by us also play an important role in ex vivo expansion of progenitor cells. The dynamics of cytokine production in co-culture and non-co-culture systems during ex vivo expansion of progenitor cells and the cytokines' effects on the ex vivo expansion remain to be determined.

Co-transplantation of adult MSC has been shown to enhance engraftment of hematopoietic stem cells in a fetal sheep model. Our results confirm that co-transplantation of MSPCs and progenitor cells from human UCB also result in accelerated rapid, medium-term and long-term engraftment of hematopoietic stem/progenitor cells.

Without wishing to be bound by any particular theory, a population of MSPCs was isolated from UCB. These cells possess some of the same morphologic, immunophenotypic, and functional characteristics of adult bone marrow-derived MSC. MSPCs from UCB could provide an alternative approach for establishment and manipulation of ex vivo expansion of progenitor cells and for differentiation of pluripotent cells.

### EXAMPLE 2

Enhanced in Vivo Homing of Uncultured and Selectively Amplified Cord Blood CD34⁺Cells by Co-transplantation with Cord Blood Derived USSCs.

USSCs exhibit an intrinsic and directable potential to develop into mesodermal, endodermal, and ectodermal fates. USSCs were evaluated for their ability to influence the homing of cord blood-derived CD34⁺ cells into the marrow and spleen of NOD/SCID mice. Cultured USSCs were co-transplanted with freshly isolated, CFDA labeled cord blood CD34⁺cells, into sublethally irradiated NOD/SCID mice. Femurs and spleens were harvested 16 hrs thereafter and the percentage of CD34⁺cells determined by flow cytometry.

USSCs induced a significant enhancement of CD34⁺ cell homing to both bone marrow and spleen (2.2±0.3 and 2.4±0.6 -fold, respectively; p<0.05). Similar findings were obtained with frozen USSC samples that had been thawed prior to transplantation. Enhanced homing by USSCs was unaltered by extensive culture passaging of the cells, with a similar degree of enhancement observed for both early (p5) and late (p10) passage USSCs.

USSCs were also found to enhance the homing of day 14 cells harvested from cultures of selectively amplified™, ex-vivo expanded cord blood lineage-negative (Lin⁻) cells. Co-transplantation of USSCs with either cultured or unmanipulated cells, did not influence the relative proportion of CD34⁺ cells which had homed to either the marrow or the spleen of NOD/SCID mice. Furthermore, the effect of USSCs was specific, as no homing enhancement could be observed by co-transplantation of CD34⁺ cells with non-cultured lineage-positive cells collected during the pre- or mid-culture (7-day) selection of Lin⁻ cells.

Demonstrated herein is the use of USSCs to enhance the homing of cord blood hematopoietic stem cells and facilitate engraftment under conditions of limiting cord blood stem cell numbers.

### OTHER EMBODIMENTS

All publications and patent applications mentioned in this specification are herein incorporated by reference to the same extent as if each independent publication or patent application was specifically and individually indicated to be incorporated by reference.

### EXAMPLE 3

Transplantation of Hematopoietic Stem/Progenitor (HSPCs) from Human Umbilical Cord Blood (hUCB), Expanded Using Mesenchymal Stem/Progenitor Cells (MSPCs) as Feeder Layers, into NOD/SCID Mice

Nonobese diabetic/severe combined immunodeficiency disease (NOD/SCID) mice were used to analyze the engraftment and differentiation of hematopoietic stem/progenitor cells (HSPCs) from human umbilical cord blood (hUCB), expanded using mesenchymal stem/progenitor cells (MSPCs) from hUCB as feeder layers, in NOD/SCID mice after transplantation.

Eight-week-old male NOD/SCID mice, weighting 19-24 g, were obtained from the Central Institute for Experimental Animals in Zhejiang Academy of Medical Sciences, and maintained in the defined flora animal facility. All animals were handled under sterile conditions.

hUCB collection was kindly provided by the Obstetrics Department of the Zhejiang Gynecological and Obstetric Hospital, Hangzhou. Parturient women gave written consent for the use of hUCB for research purposes. Collected hUCB was hepatinized.

Mononuclear cells (MNCs) from hUCB were isolated and resuspended in IMDM with 20% FBS and 100 ng/mL each of recombinant human stem cell factor (rhSCF) recombinant human granulocyte colony-stimulating factor (rhG-CSF) and recombinant human megakaryocyte growth and development factor (rhMGDF) (Amgen Inc., Thousand Oaks, Calif.), and then seeded into 25-cm² flasks with or without the hUCB mesenchymal stem/progenitor adherent cells. The MNCs were cultured in 100% humidified 5% CO₂ in air at 37 C for 14 days. At the beginning (the first day) and the ending (the 14th day) of culture, nonadherent cells in the culture medium were assayed for colony-forming cells in complete methylcellulose without erythropoietin (Gencyte, Anherat, N.Y.) as the progenitor cell assay.

Transplantation into NOD/SCID Mice

At 24 h before transplantation, male NOD/SCID mice at 8 weeks of age were irradiated with a ⁶⁰Coγ in a split dose with a 4 h interval between doses. Each dose is 15 min at 14.5 r/min.

The expanded HSPCs were transplanted by tail-vein injection into lethally irradiated NOD/SCID mice. Group A of 15 irradiated mice was used for transplantations of cells expanded in non-coculture scheme, group B of 15 irradiated mice for transplantation of cells expanded in coculture scheme, and group C of 15 irradiated mice as control by being injected with the same volume (200 μL) of PBS/1% HSA. Each group was performed in triplicate. The number of transplanted cells for each irradiated mouse was 8.8×10⁶ cells in 200 μL suspension.

### Hematopoietic Reconstruction of NOD/SCID Mice

Since the second day after transplantation, 100 μL peripheral blood was collected by retro orbital sampling from each mouse at regular intervals. Peripheral blood was diluted up to 250 μL with PBS/1% HSA, and then analyzed for hematopoietic recovery using Advia 120 (Bayer, Leverkusen, Germany).

Polymerase chain reaction (PCR) amplification of human Alu repetitive sequence gene was employed for testing the presence of human cells in the NOD/SCID mice that had received transplants. Mice were killed at 8 weeks after transplantation, and the bone marrow (from the femurs and tibiae) and peripheral blood were harvested. Genetic DNA was isolated from bone marrow cells and peripheral blood cells. Genetic DNA isolated from hUCB cells was used as positive control, and genetic DNA isolated from the untreated NOD/SCID mice bone marrow cells as negative control. PCR amplification was performed as following: the sequences of primers were 5′-CTG GGC GA C AGA ACG AGA TTC TAT-3′ and 5′-CTC ACT ACT TGG TGA CAG GTT CA-3′. Reaction system (50 μL): 5 μL 10×amplification buffer, 3 μL 25 mmol/L MgCl₂, 2.5 U Taq DNA polymerase, mmol/L dNTP 0.2, 0.25 μmol/L each primer, and 0.2 μg template DNA. The amplification conditions were as follows: pre-denaturation at 95° C./5 min, denaturation at 95° C./30 s, annealing at 54° C./45 s and extension at 72° C./45 s during 30 cycles, finally extended at 72° C. for 5 min. The amplification product was visualized as a 224-bp band on 2% agarose gel electrophoresis and ethidium bromide staining.

The irradiated mice without transplantation of expanded cells (group C) were dead within 13 days after irradiation. For the irradiated mice transplanted with expanded cells in non-coculture scheme (group A), three mice died respectively at the 12th, 16th and 17th day after transplantation. For the irradiated mice transplanted with expanded cells in coculture scheme (group B), one mouse died at 23th day after transplantation. No death was observed in the following time for these two transplantation groups.

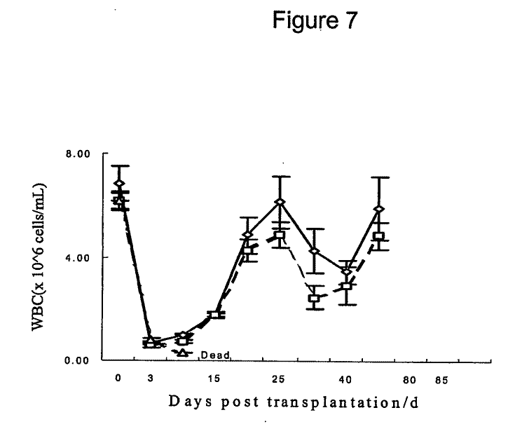

The number of white blood cells (WBC) in groups transplanted with expanded cells began to increase at the 15th day after transplantation. The leucocytes number reached baseline value at the 25th day after transplantation, and then decreased distinctly. The number returned to the baseline value again till the 45-55th days after transplantation. (FIG. 7).

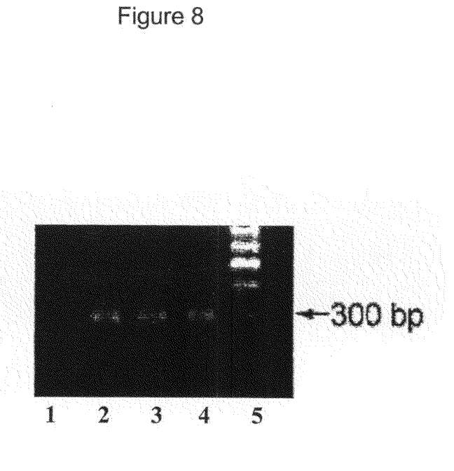

Polymerase chain reaction (PCR) amplification of human Alu repetitive sequence gene was employed for testing the presence of human cells in the NOD/SCID mice that had received transplants (FIG. 8). Human Alu repetitive sequence gene was detected in 12 out of 14 NOD/SCID mice that survived after transplantation with expanded cells in coculture scheme. For non-coculture scheme, human Alu repetitive sequence gene was detected in 9 out of 12 NOD/SCID mice survived after transplantation. The positive percentage was 85.7% for coculture scheme and 75.0% for non-coculture scheme. The Alu repetitive sequence was not detected in NOD/SICD mice without transplantation.

Hematopoietic stem/progenitor cells (HSPCs) from human umbilical cord blood (hUCB) were expanded using mesenchymal stem/progenitor cells (MSPCs) from hUCB as feeder layers, and then transplanted these expanded cells into the lethally irradiated NOD/SCID mice to assess the effects of expanded cells on hematopoietic recovery. It was showed that the coculture scheme increased ex vivo expansion of CB-derived HSPCs more effectively than non-coculture scheme, and MSCs were propitious to expansion of HSPCs as feeder layers.

WBC numbers in mice transplanted with expanded cells in both coculture scheme and non-coculture scheme reached the baseline value rapidly (began to increase at 15th day and reached the peak at 25th day after transplantation). Expanded cells from the coculture scheme appeared to be more favorable for the second increasing of WBCs and reconstructed hematopoiesis more rapidly in the transplanted mice.

In further embodiments, the dose of irradiation is listed as a split dose of may range-from about 300 to 400 rads for NOD/SCID mice. In certain embodiments, the total WBC recovery may include chimerism on each count to know the percentage human cells.

As used herein, “sublethally or lethally irradiated mice” include mice that die after a period of time, for example 7 days post irradiation, or from between about 1-30 days post irradiation, or about 7-16 days post irradiation.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

1. A method for culturing nucleated cells from umbilical cord blood (UCB) comprising preparing a co-culture comprising said nucleated cells and adherent stroma cells from UCB, wherein said nucleated cells and said adherent stroma cells are added separately to said co-culture.

2. The method of claim 1, wherein said nucleated cells comprise immature or mature cells.

3. The method of claim 2, wherein said immature cells comprise progenitor cells.

4. The method of claim 2, wherein said mature cells are selected from granulocytes, neutrophils, megakaryocytes, macrophages, T cells, natural killer cells, and red blood cells.

5. The method of claim 1, wherein said nucleated cells are selected from CD34⁻ cells, CD34⁺ cells, or Lin⁻ cells.

6. The method of claim 1, wherein said adherent stroma cells comprise mesenchymal stem cells.

7. The method of claim 1, wherein said method yields a greater number of nucleated cells than would be present in a culture of nucleated cells grown in the absence of said adherent stroma cells.

8. The method of claim 6, wherein said mesenchymal stem cells are unrestricted somatic stem cells (USSCs).

9. The method of claim 8, wherein said USSCs are characterized as being CD13⁺, CD29⁺, CD90⁺, CD105⁺, CD166⁺, SH2⁺, SH3⁺, SH4⁺, CD45⁻, CD34⁻, and CD14⁻.

10. The method of claim 9, wherein said USSCs are further characterized as expressing fibulin-2 and lacking expression of hyaluronan synthase and fibromodulin

11. The method of claim 1, wherein said nucleated cells or said adherent stroma cells are isolated from UCB or derived from cells isolated from UCB.

12. The method of claim 1, wherein, prior to said culturing, said nucleated cells or adherent stroma cells are substantially separated from other cell types.

13-42. (canceled)

43. A method for increasing engraftment of donor nucleated cells in a subject, comprising administering a composition comprising nucleated cells from umbilical cord blood (UCB) and adherent stroma cells from UCB to said subject.

44. The method of claim 43, wherein said nucleated cells comprise immature or mature cells.

45. The method of claim 44, wherein said immature cells comprise progenitor cells.

46. The method of claim 44, wherein said mature cells are selected from granulocytes, neutrophils, megakaryocytes, macrophages, T cells, natural killer cells, and red blood cells.

47. The method of claim 43, wherein said nucleated cells are selected from CD34⁻ cells, CD34⁺ cells, and Lin⁻ cells.

48. The method of claim 43 wherein said adherent stroma cells comprise mesenchymal stem cells.

49. The method of claim 43, wherein said method yields a greater number of engrafting cells relative to the number of engrafting cells in a composition lacking said adherent stroma cells.

50. The method of claim 48, wherein said mesenchymal stem cells are unrestricted somatic stem cells (USSCs).

51. The method of claim 50, wherein said USSCs are characterized as being CD13⁺, CD29⁺, CD90⁺, CD105⁺, CD166⁺, SH2⁺, SH3⁺, SH4⁺, CD45⁻, CD34⁻, and CD14⁻.

52. The method of claim 51, wherein said USSCs are further characterized as expressing fibulin-2 and lacking expression of hyaluronan synthase and fibromodulin

53. The method of claim 43, wherein said nucleated cells or said adherent stroma cells are isolated from UCB or derived from cells isolated from UCB.

54-60. (canceled)

61. A composition comprising nucleated cells and adherent stroma cells, wherein said nucleated cells and adherent stroma cells are obtained from umbilical cord blood (UCB), and wherein said composition is suitable for infusion or engraftment into a subject.

62. The composition of claim 61, wherein said adherent stroma cells comprise mesenchymal stem cells.

63-69. (canceled)
